(19) United States
(12) Patent Application Publication
(10) Pub. No.: US 2016/0228016 A1
(43) Pub. Date: Aug. 11, 2016

(54) MEDICAL SENSOR ASSEMBLY AND MOUNTING ASSEMBLY THEREFOR

(71) Applicant: ELCAM MEDICAL AGRICULTURAL COOPERATIVE ASSOCIATION LTD., Merom Ha-Galil (IL)

(72) Inventors: Ziv NAFTALOVITZ, Kibbutz Eilon (IL); Yuval DAGAN, Moshav Gilon (IL); Nadav SHLOMOT, Merom Ha Galil (IL)

(73) Assignee: ELCAM MEDICAL AGRICULTURAL COOPERATIVE ASSOCIATION LTD., Merom Ha-Galil (IL)

(21) Appl. No.: 15/022,241

(22) PCT Filed: Sep. 17, 2014
(86) PCT No.: PCT/IL14/50835

§ 371 Date: Mar. 16, 2016

Related U.S. Application Data
(60) Provisional application No. 61/878,724, filed on Sep. 17, 2013.

(51) Int. Cl. A61B 5/021; A61B 5/03

(52) U.S. Cl. CPC A61B 5/02141; A61B 5/03; A61B 2562/227; A61B 2562/18; A61B 2562/0247; A61B 2562/16

(57) ABSTRACT

A pressure transducer assembly including a mounting assembly including an electrical connector configured for selectable galvanic engagement with a pressure transducer and a medical sensor assembly including a pressure transducer, the pressure transducer including a flow pathway and a pressure sensor in pressure sensing communication with the flow pathway, the pressure sensor including a plurality of electrical contacts configured for direct galvanic engagement with the electrical connector only when the medical sensor assembly is mounted in operative engagement with the mounting assembly.

### REFERENCE TO RELATED APPLICATION

Reference is hereby made to U.S. Provisional Patent Application No. 61/878,724, filed Sep. 17, 2013 and entitled MEDICAL SENSOR AND A MOUNTING DEVICE THEREFOR, the disclosure of which is hereby incorporated by reference and priority of which is hereby claimed pursuant to 37 CFR 1.78(a)(4) and (5)(i).

### FIELD OF THE INVENTION

The present invention relates to medical sensors and mounting assemblies therefor.

### BACKGROUND OF THE INVENTION

Various types of medical sensors and mounting assemblies therefor are known.

### SUMMARY OF THE INVENTION

The present invention seeks to provide an improved medical sensor and mounting assembly therefor.

There is thus provided in accordance with an embodiment of the present invention a pressure transducer assembly including a mounting assembly including an electrical connector configured for selectable galvanic engagement with a pressure transducer and a medical sensor assembly including a pressure transducer, the pressure transducer including a flow pathway and a pressure sensor in pressure sensing communication with the flow pathway, the pressure sensor including a plurality of electrical contacts configured for direct galvanic engagement with the electrical connector only when the medical sensor assembly is mounted in operative engagement with the mounting assembly.

Preferably, the electrical connector is configured to be moved by engagement with the medical sensor assembly from a first non-electrically connected operative orientation to a second electrically connected operative orientation. Additionally, the medical sensor assembly also includes an electrical connector engagement element operative to move the electrical connector from the first non-electrically connected operative orientation to the second electrically connected operative orientation upon engagement of the medical sensor assembly with the mounting assembly.

In accordance with a preferred embodiment of the present invention the pressure transducer assembly also includes a cover element for preventing fluid contact with the electrical connector when the mounting assembly is not in engagement with the medical sensor assembly. Additionally, the mounting assembly also includes an upper housing portion and the cover element is in a protective position with respect to the upper housing portion thereby preventing the fluid contact with the electrical connector when the mounting assembly is not in engagement with the medical sensor assembly.

Preferably, the medical sensor assembly slidingly engages the mounting assembly to move the electrical connector from the first non-electrically connected operative orientation to the second electrically connected operative orientation. Additionally or alternatively, the plurality of electrical contacts of the pressure sensor are exposed when the medical sensor assembly is not engaged with the mounting assembly.

In accordance with a preferred embodiment of the present invention the medical sensor assembly includes an aperture for providing access to the plurality of electrical contacts of the pressure sensor by the electrical connector for the selectable direct galvanic engagement.

There is also provided in accordance with another preferred embodiment of the present invention a medical sensor assembly useful with a mounting assembly having an electrical connector arranged for selectable galvanic engagement with the medical sensor assembly, the medical sensor assembly including a pressure transducer including a flow pathway and a pressure sensor in pressure sensing communication with the flow pathway, the pressure sensor including a plurality of exposed electrical contact pads configured for direct galvanic engagement with the electrical connector when the medical sensor assembly is mounted in operative engagement with the mounting assembly.

Preferably, the exposed electrical contact pads are configured for direct galvanic engagement with the electrical connector only when the medical sensor assembly is mounted in operative engagement with the mounting assembly.

In accordance with a preferred embodiment of the present invention the electrical connector is configured to be moved by engagement with the medical sensor assembly from a first non-electrically connected operative orientation to a second electrically connected operative orientation. Additionally, the medical sensor assembly also includes an electrical connector engagement element operative to move the electrical connector from the first non-electrically connected operative orientation to the second electrically connected operative orientation. Additionally or alternatively, the medical sensor assembly slidingly engages the mounting assembly to move the electrical connector from the first non-electrically connected operative orientation to the second electrically connected operative orientation.

Preferably, the medical sensor assembly includes an aperture for providing access to the plurality of exposed electrical contact pads of the pressure sensor by the electrical connector for the selectable direct galvanic engagement.

There is further provided in accordance with yet another preferred embodiment of the present invention a mounting assembly for use with a medical sensor assembly, the medical sensor assembly including a pressure transducer, the mounting assembly including an electrical connector configured for selectable direct galvanic engagement with the pressure transducer and a cover element for preventing fluid contact with the electrical connector when the mounting assembly is not in engagement with the medical sensor assembly.

Preferably, the mounting assembly also includes an upper housing portion which cooperates with the cover element, the cover element being in electrical connector protective engagement in cooperation with the upper housing portion when the mounting assembly is not in engagement with the medical sensor assembly.

In accordance with a preferred embodiment of the present invention the electrical connector is configured to be moved by engagement with the medical sensor assembly from a first non-electrically connected operative orientation to a second electrically connected operative orientation.

Preferably, the mounting assembly is configured for sliding engagement with the medical sensor assembly.

There is yet further provided in accordance with still another preferred embodiment of the present invention a method for removably interconnecting a pressure transducer assembly and a monitor, the method including providing a pressure transducer assembly including a medical sensor assembly, the medical sensor assembly including a pressure transducer having a plurality of electrical contacts, and a mounting assembly, the mounting assembly including a first electrical connector configured for selectable direct galvanic engagement with the plurality of electrical contacts and a second electrical connector arranged to be connected to the monitor and to the first electrical connector, positioning the mounting assembly onto a fixed support, electrically connecting the second electrical connector with the monitor and mounting the medical sensor assembly onto the mounting assembly, such that the plurality of electrical contacts of the pressure sensor automatically establish direct galvanic engagement with the first electrical connector when the medical sensor assembly is mounted in operative engagement with the mounting assembly.

Preferably, the method also includes moving the first electrical connector from a first non-electrically connected operative orientation to a second electrically connected operative orientation.

In accordance with a preferred embodiment of the present invention the mounting the medical sensor assembly onto the mounting assembly includes slidingly displacing the medical sensor assembly in engagement with the mounting assembly. Additionally or alternatively, the mounting the medical sensor assembly onto the mounting assembly includes linearly displacing the medical sensor assembly into engagement with the mounting assembly.

### BRIEF DESCRIPTION OF THE DRAWINGS

The present invention will be understood more fully from the following detailed description, taken in conjunction with the drawings in which:

FIGS. 1A & 1B are, respectively, simplified pictorial illustrations of a mounting assembly and a medical sensor assembly mounted onto the mounting assembly, in accordance with a preferred embodiment of the present invention;

FIG. 2 is a simplified partially exploded view pictorial illustration of the apparatus of FIG. 1B;

FIGS. 3A, 3B, 3C, 3D and 3E are, respectively, simplified pictorial, planar bottom view, planar top view, planar side view and planar end view illustrations of a base element forming part of the mounting assembly shown in FIGS. 1A-2;

FIGS. 4A, 4B, 4C, 4D and 4E are, respectively, simplified pictorial, planar bottom view, planar top view, planar side view and planar end view illustrations of a lower housing element forming part of the mounting assembly shown in FIGS. 1A-2;

FIGS. 5A, 5B, 5C, 5D, 5E and 5F are, respectively, simplified pictorial, planar side view, first planar end view, planar top view, planar bottom view and second planar end view illustrations of a moveable cover element forming part of the mounting assembly shown in FIGS. 1A-2;

FIGS. 6A, 6B, 6C and 6D are, respectively, simplified pictorial, planar top view, planar side view and planar end view illustrations of a rotatable electrical contact element forming part of the mounting assembly shown in FIGS. 1A-2;

FIGS. 7A, 7B, 7C and 7D are, respectively, simplified pictorial, planar bottom view, planar top view and sectional side view illustrations of an upper housing element forming part of the mounting assembly shown in FIGS. 1A-2, the side sectional view being taken along lines D-D in FIG. 7C;

FIGS. 8A, 8B, 8C and 8D are, respectively, simplified pictorial, planar top view, planar bottom view and sectional side view illustrations of a medical sensor assembly, forming part of the mounting assembly shown in FIGS. 1A-2, the side sectional view being taken along lines D-D in FIG. 8B;

FIG. 9 is a simplified exploded view illustration of the medical sensor assembly of FIGS. 8A-8D;

FIGS. 10A, 10B, 10C, 10D, 10E and 10F are, respectively, simplified pictorial, planar bottom view, planar top view, planar side view, planar end view and internal side sectional view illustrations of a base element forming part of the medical sensor assembly shown in FIGS. 1A-2, 8A-8D and 9, the internal side sectional view being taken along lines F-F in FIG. 10C;

FIGS. 11A, 11B, 11C and 11D are respectively, simplified pictorial, planar top view, planar bottom view and sectional side view illustrations of a medical sensor assembly, forming part of the mounting assembly shown in FIGS. 1A-2, the side sectional view being taken along lines D-D in FIG. 11B;

FIG. 12 is a simplified exploded view illustration of the medical sensor assembly of FIGS. 11A-11D;

FIGS. 13A, 13B, 13C, 13D, 13E and 13F are respectively, a planar top view and five sectional simplified illustrations of the mounting assembly of FIGS. 1A-2, the sectional illustrations being taken along respective lines B-B, C-C and D-D in FIG. 13A, lines E-E in FIG. 13C and lines F-F in FIG. 13D;

FIGS. 14A and 14B are simplified pictorial top view and side view illustrations of the medical sensor assembly of FIGS. 11A-13 in a first operative orientation;

FIG. 14C is a simplified sectional illustration of the medical sensor assembly of FIGS. 11A-14B in a first operative orientation taken along section lines C-C in FIG. 14A;

FIGS. 15A and 15B are simplified pictorial top view and side view illustrations of the medical sensor assembly of FIGS. 11A-14C in a second operative orientation;

FIG. 15C is a simplified sectional illustration of the medical sensor assembly of FIGS. 11A-15B in a second operative orientation taken along section lines C-C in FIG. 15A;

FIGS. 16A and 16B are simplified pictorial top view and side view illustrations of the medical sensor assembly of FIGS. 11A-15D in a third operative orientation;

FIG. 16C is a simplified sectional illustration of the medical sensor assembly of FIGS. 11A-16B in a third operative orientation taken along section lines C-C in FIG. 16A;

FIGS. 17A and 17B are simplified pictorial top view and side view illustrations of the medical sensor assembly of FIGS. 11A-16D in a fourth operative orientation; and

FIGS. 17C and 17D are simplified sectional illustrations of the medical sensor assembly of FIGS. 11A-17B in a fourth operative orientation taken along respective section lines C-C and D-D in FIG. 17A.

### DETAILED DESCRIPTION OF A PREFERRED EMBODIMENT

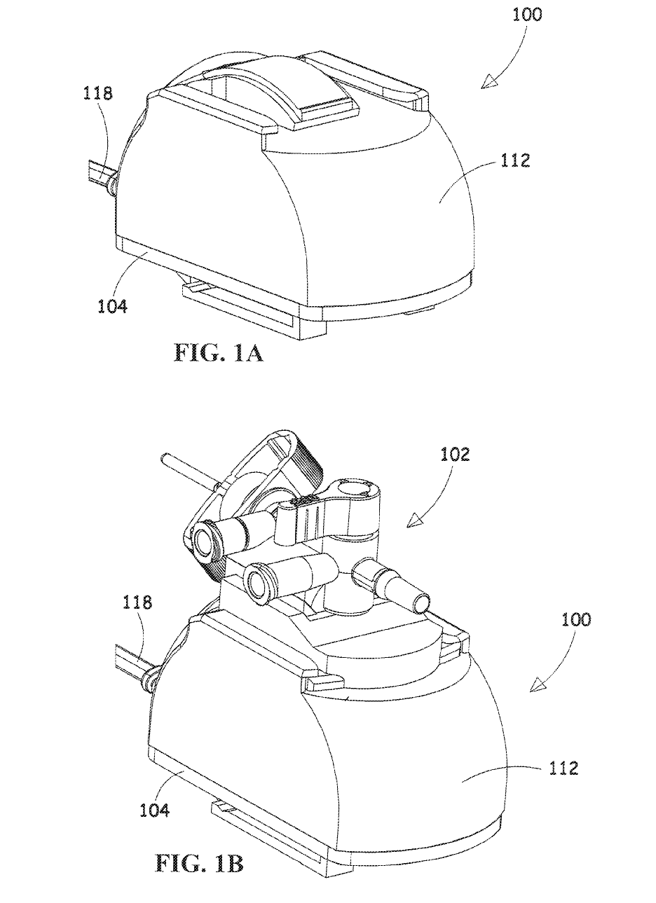

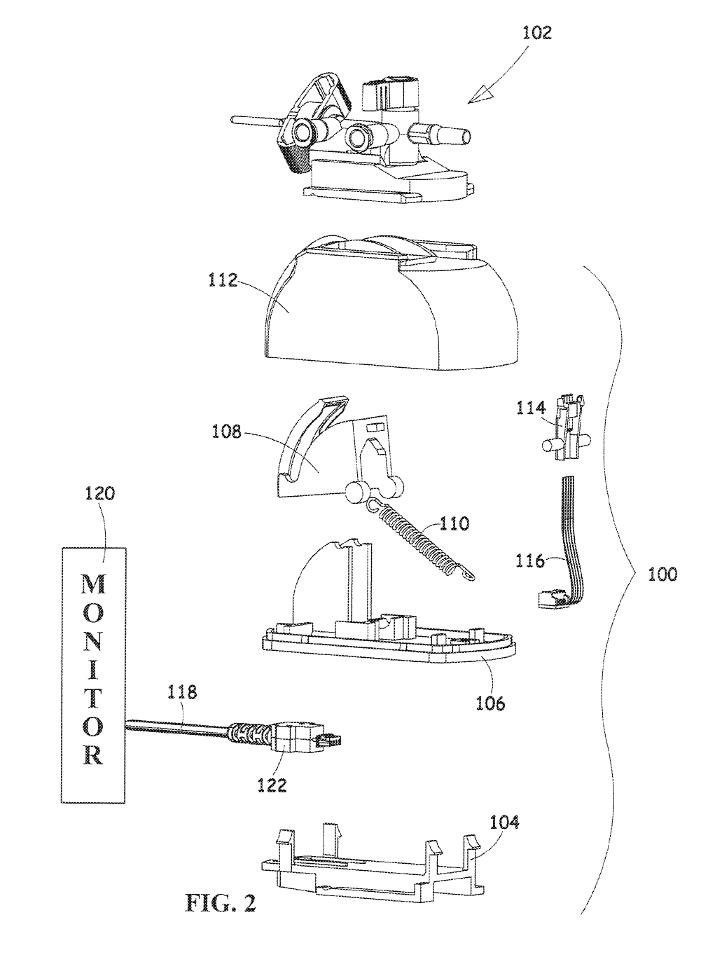

Reference is now made to FIGS. 1A & 1B, which are, respectively, simplified pictorial illustrations of a mounting assembly and a medical sensor assembly mounted onto the mounting assembly, in accordance with a preferred embodiment of the present invention, and to FIG. 2, which is a simplified partially exploded view pictorial illustration of the apparatus of FIG. 1B. It is appreciated that FIGS. 1A, 1B & 2 are examples of a pressure transducer assembly having multiple novel features. It is appreciated that other examples of pressure transducer assemblies may exist in which only one or more of the novel features of the present invention may be present.

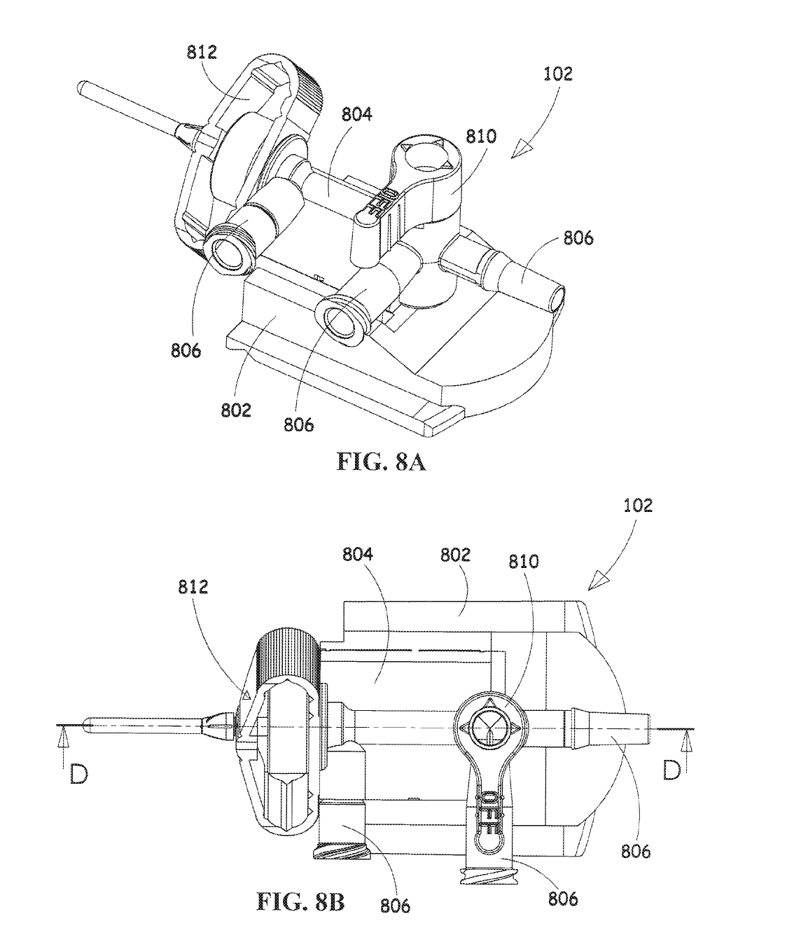

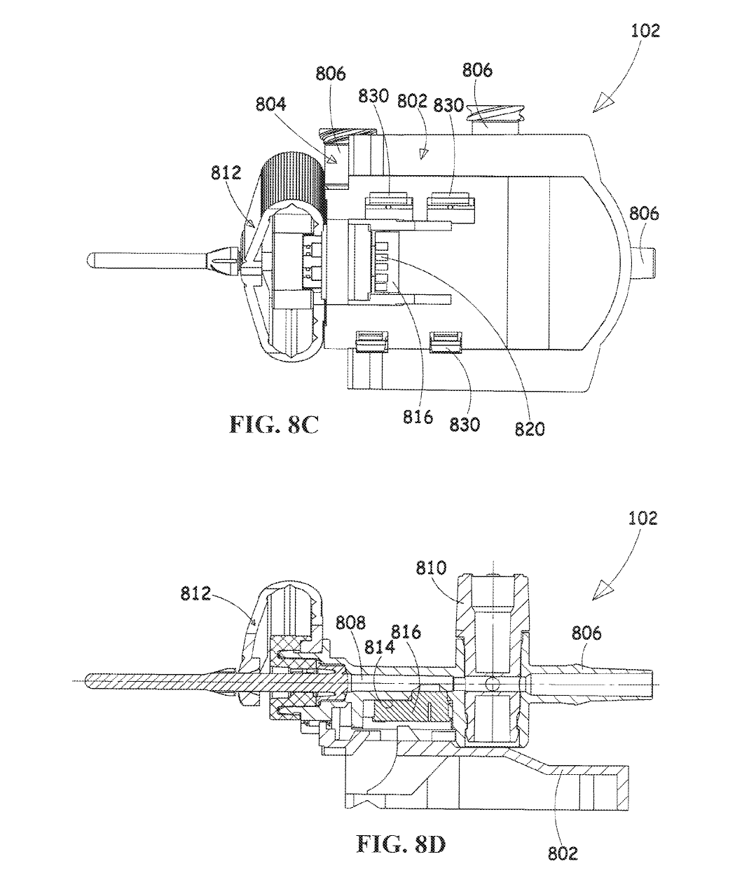

As seen in FIGS. 1A, 1B & 2, there is provided a mounting assembly 100 and a medical sensor assembly 102 removably mounted thereon. The medical sensor assembly 102 is similar in its overall structure and operation to a Disposable Pressure Transducer 650101, commercially available from Elcam Medical, Kibbutz Baram, 15 Israel, and is generally constructed and operative in a manner described and claimed in U.S. Pat. No. 6,511,434, the disclosure of which is hereby incorporated by reference, and is modified as described with particularity hereinbelow with reference to FIGS. 8A-9 and 11A-12. The medical sensor assembly 102 is typically employed to measure blood pressure of a patient but may be employed for measuring any other body pressure of a patient, such as intra-abdominal pressure.

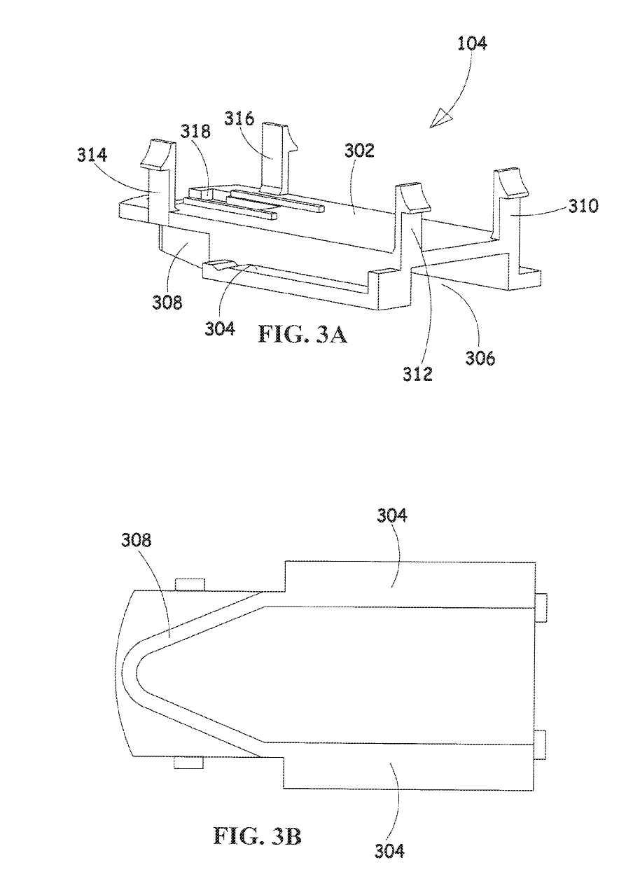

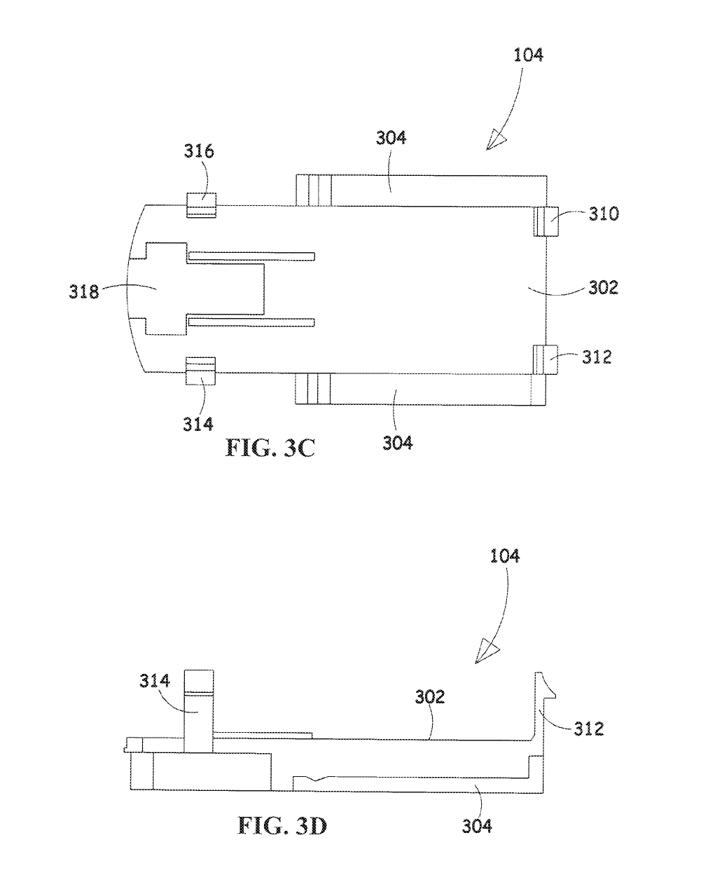

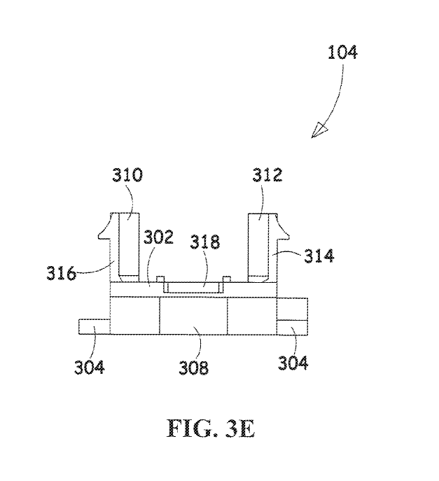

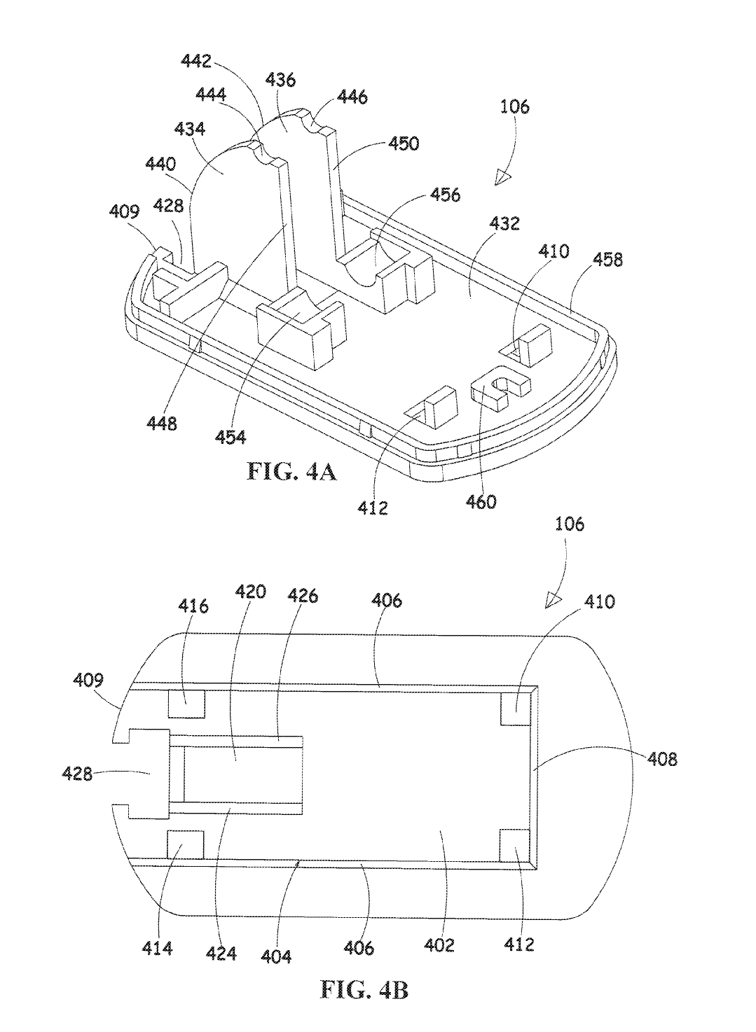

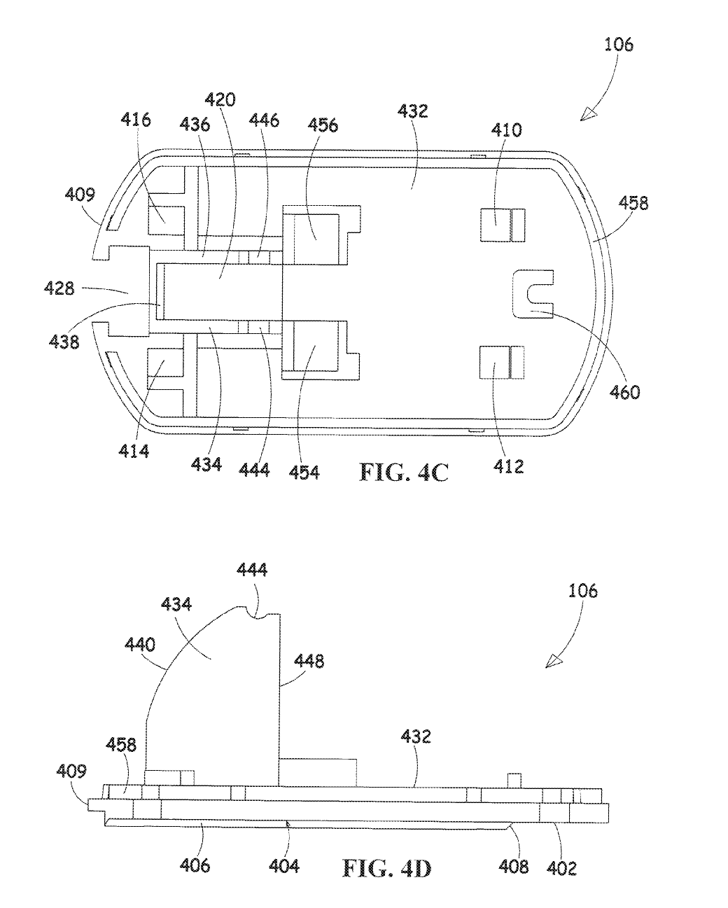

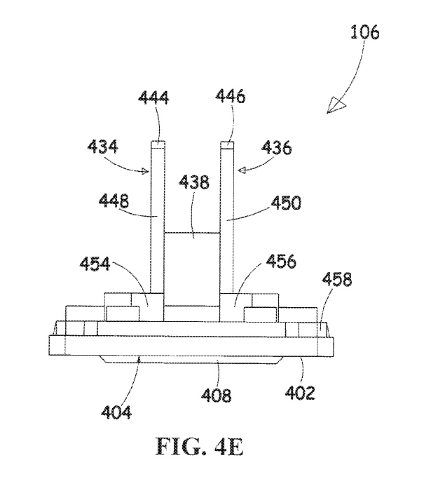

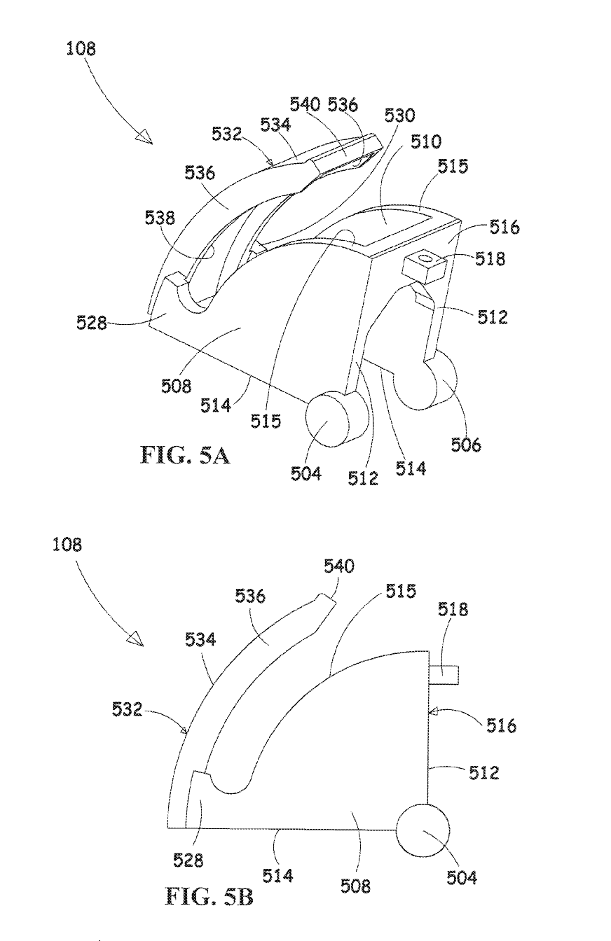

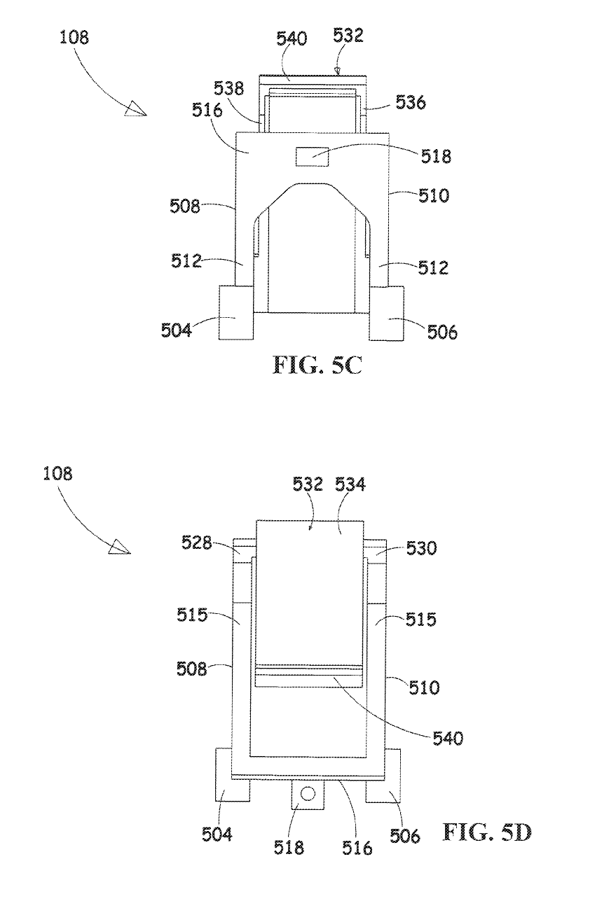

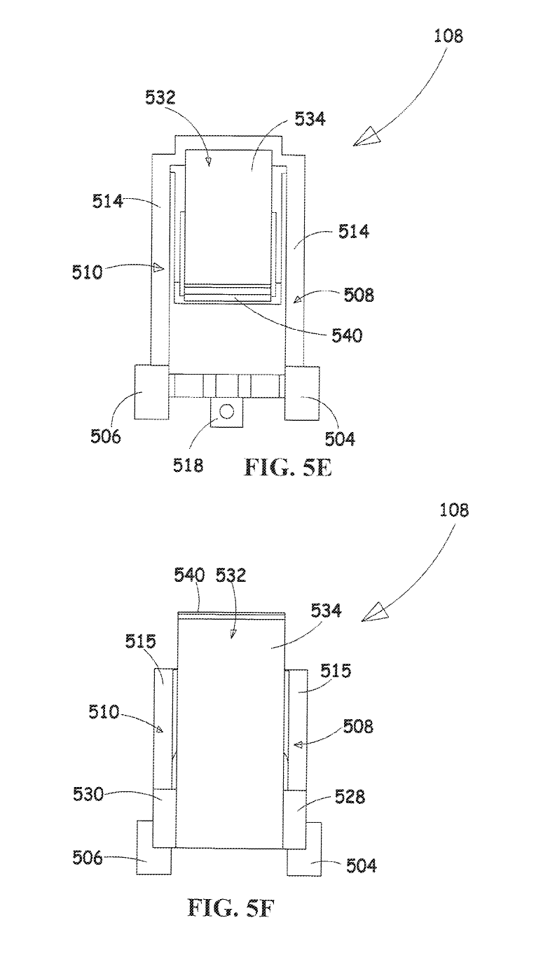

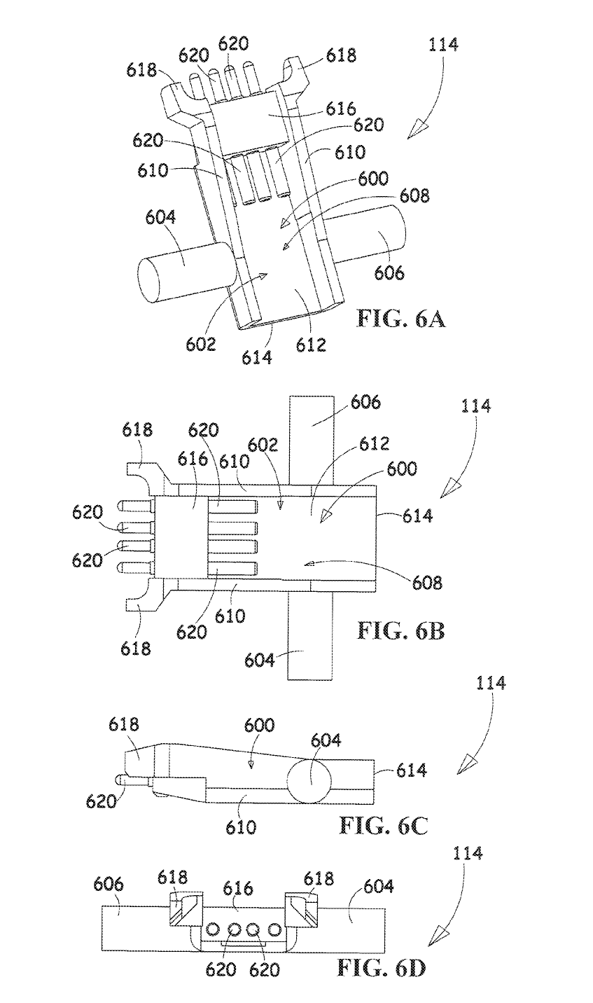

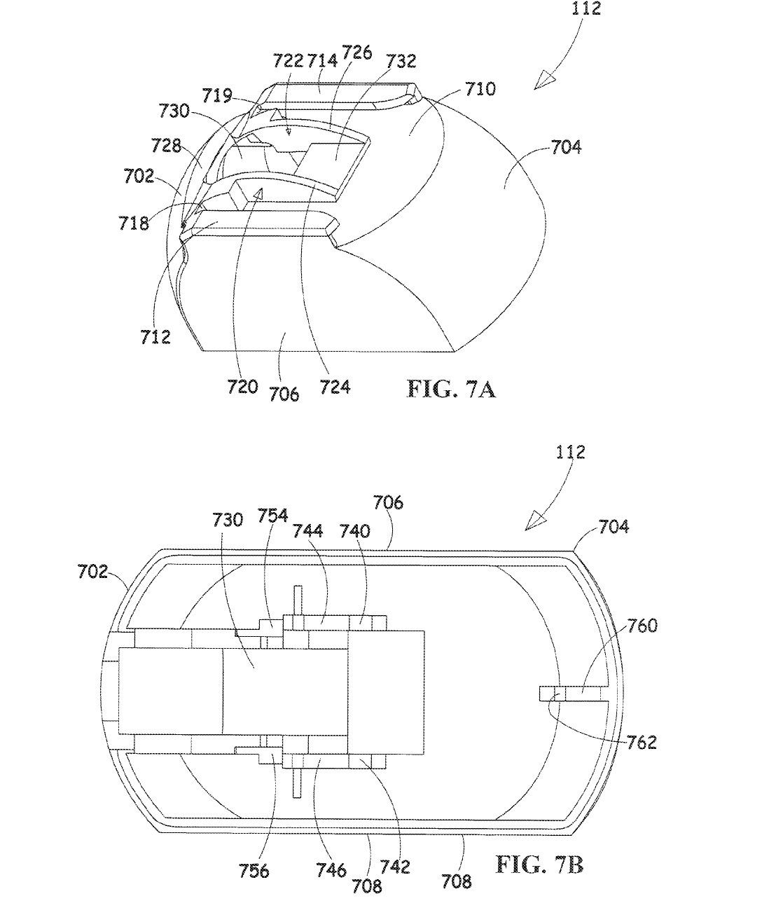

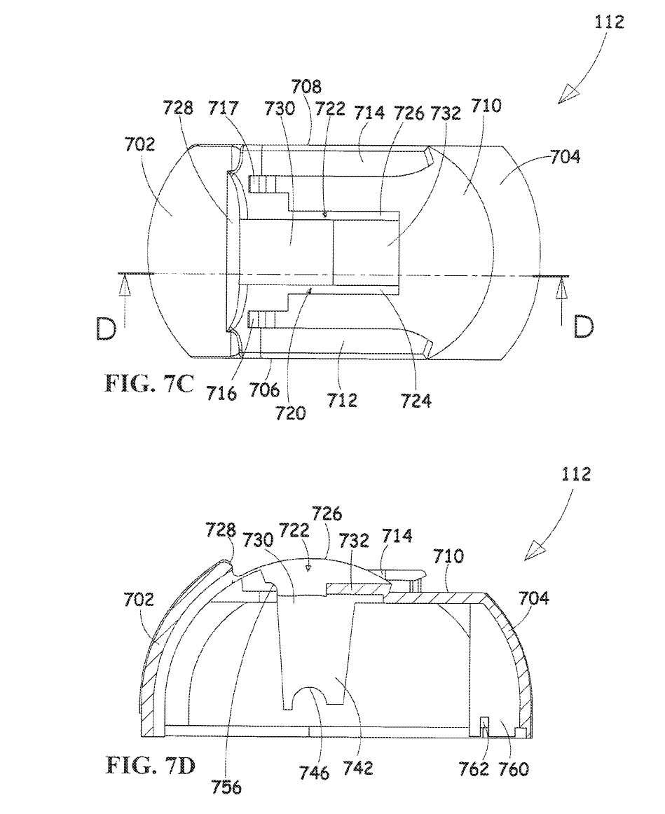

As seen particularly in FIG. 2, the mounting assembly 100 preferably comprises a base element 104, which is described hereinbelow with reference to FIGS. 3A-3E, onto which is mounted a lower housing element 106, which is described hereinbelow with reference to FIGS. 4A-4E. Preferably, a movable cover element 108, described hereinbelow with reference to FIGS. 5A-5F, and associated spring 110 are mounted onto lower housing element 106 and are generally enclosed by an upper housing element 112, described hereinbelow with reference to FIGS. 7A-7D. A rotatable electrical contact element 114, described hereinbelow with reference to FIGS. 6A-6D, is also generally enclosed within upper housing element 112, as is a flexible connector 116, which is electrically coupled to rotatable electrical contact element 114 and which is in turn connected to a conventional monitor connection cord assembly 118, and thereby to a monitor 120, such as a Dash 400, commercially available from General Electric. Monitor connection cord assembly 118 preferably includes an electrical contact end portion 122, which is electrically coupled to an end of flexible connector 116.

Throughout the description of mounting assembly 100, the side of the mounting assembly 100 from which the monitor cord assembly 118 exits is referred to as the forward side or forward facing side, and the side distant therefrom is referred to as the rearward side or rearward facing side.

Reference is now made to FIGS. 3A, 3B, 3C, 3D and 3E, which are respectively, simplified pictorial, planar bottom view, planar top view, planar side view and planar front end view illustrations of base element 104, forming part of the mounting assembly shown in FIGS. 1A-2. As seen in FIGS. 3A-3E, the base element 104 preferably has a central, raised generally planar surface 302 bounded on two sides by elongate foot portions 304 lying below surface 302 and defining therewith a bottom facing recess 306. Recess 306 is open at one end of base element 104 and is bounded by foot portions 304 and by a joining portion 308, which joins foot portions 304. Four upstanding toothed engagement protrusions 310, 312, 314 and 316 extend upwardly adjacent four corners of surface 302 for snap-fit engagement with the lower housing element 106. A monitor connection cord end retaining recess 318 is formed adjacent one end of surface 302.

Elongate foot portions 304 are preferably configured for slidingly positioning mounting assembly 100 onto a fixed support (not shown) prior to use.

Reference is now made to FIGS. 4A, 4B, 4C, 4D and 4E, which are, respectively, simplified pictorial, planar bottom view, planar top view, planar side view and planar rear end view illustrations of a lower housing element forming part of the mounting assembly shown in FIGS. 1A-2.

As seen in FIGS. 4A-4E, and particularly in FIG. 4B, the lower housing element 106 preferably has a generally planar bottom facing surface 402 having formed thereon a rectangular outline protrusion 404 having generally parallel side portions 406, a closed end 408 and an open end 409. Located within generally rectangular outline 30 protrusion 404 are four throughgoing apertures 410, 412, 414 and 416 for accommodating upstanding toothed engagement protrusions 310, 312, 314 and 316, respectively, of the base element 104. Extending between apertures 414 and 416 and toward closed end 408 is a central throughgoing aperture 420 having upwardly inwardly tapered side surfaces 424 and 426. Forwardly of aperture 420 is a throughgoing cut out 428 for accommodating an end of monitor connection cord assembly 118.

As seen in FIGS. 4A-4E, and particularly in FIG. 4C, the lower housing element preferably has a generally planar top facing surface 432, from which extend a pair of generally mutually parallel, mutually spaced upstanding portions 434 and 436 which are located above respective side surfaces 424 and 426. Upstanding portions 434 and 436 are joined by a transverse upstanding portion 438 which extends above an imaginary line that separates aperture 420 from cut out 428. As seen particularly in FIG. 4A, upstanding portions 434 and 436 define respective curved side and top edge surfaces 440 and 442 and a pair of respective mutually axially aligned pivot support edge surfaces 444 and 446 as well as respective vertical edges 448 and 450.

Adjacent vertical edges 448 and 450, and slightly raised with respect to generally planar top facing surface 432, there are provided a pair of mutually axially aligned pivot support cylindrical surfaces 454 and 456.

Also extending upwardly from generally planar top facing surface 432 is a generally circumferential protrusion 458 and a spring attachment protrusion 460.

Reference is now made to FIGS. 5A, 5B, 5C, 5D, 5E and 5F, which are respectively, simplified pictorial, planar side view, planar rear end view, planar top view, planar bottom view and planar front end view illustrations of a moveable cover element forming part of the mounting assembly shown in FIGS. 1A-2.

As seen in FIGS. 5A-5F, the moveable cover element 108 preferably comprises a pair of generally cylindrical pivot portions 504 and 506 which are arranged for pivotable rotation in respective mutually axially aligned pivot support cylindrical surfaces 454 and 456 of the lower housing portion 106. Pivot portions 504 and 506 are preferably attached at respective corners of generally quarter-circular side wall portions 508 and 510, each having generally mutually perpendicular edges 512 and 514, joined by a curved edge 515. Edges 512 of side wall portions 508 and 510 are joined by a partially cut out wall portion 516, which extends generally perpendicularly to side wall portions 508 and 510 and is formed with an apertured spring engagement protrusion 518.

Each of side wall portions 508 and 510 extends along edge 514 to define a pair of side supports 528 and 530 which support a curved cover portion 532 having a main curved portion 534, a pair of depending side portions 536 having downward facing edges 538 and a forward edge portion 540. It is appreciated that edges 538 are spaced from respective curved edges 515.

Reference is now made to FIGS. 6A, 6B, 6C and 6D, which are, respectively, simplified pictorial, planar top view, planar side view and planar end view illustrations of a rotatable electrical contact element, such as rotatable electrical contact element 114, forming part of the mounting assembly shown in FIGS. 1A-2.

As seen in FIGS. 6A-6D, rotatable electrical contact element 114 comprises a base element 600, preferably injection molded of plastic, including a generally rectangular portion 602 having extending outwardly therefrom and transversely thereto a pair of mutually axially aligned pivot rod portions 604 and 606, which are arranged for pivotable rotation while being supported by respective mutually axially aligned pivot support edge surfaces 444 and 446 of lower housing element 106.

Rectangular portion 602 includes a rectangular slot 608 delimited by raised side wall portions 610 and having a planar floor surface 612 extending from an open end 614 of slot 608 to a bulkhead 616. Side wall portions 610 extend alongside bulkhead 616 and each preferably terminate in a cam-driven, sensor engagement finger 618. A plurality of electrical contact pins 620 extend in mutually electrically insulated relationship through bulkhead 616 from a location slightly forward of the termination of fingers 618 to a location rearward of bulkhead 616 along and spaced from planar floor surface 612 and side wall portions 610.

Reference is now made to FIGS. 7A, 7B, 7C and 7D, which are, respectively, simplified pictorial, planar bottom view, planar top view and sectional side view illustrations of an upper housing element, such as upper housing element 112, forming part of the mounting assembly shown in FIGS. 1A-2, the side sectional view being taken along lines D-D in FIG. 7C.

As seen in FIGS. 7A-7D, upper housing element 112 comprises a generally concave element, which is preferably injection molded as one piece. Upper housing element 112 includes generally rounded forward and rearward outer facing surfaces 702 and 704, joined by generally planar outer facing side wall surfaces 706 and 708 and by a generally planar outer facing top surface 710.

Each of outer facing side wall surfaces 706 and 708 preferably terminates at the top thereof in a generally longitudinal upwardly extending and inwardly transversely extending slot defining portion, here designated respectively by reference numerals 712 and 714, which are mutually parallel and parallel to top surface 710 and define therewith and with transverse raised protrusions 716 and 717, a slidable engagement locking slot for slidable locking engagement therewith of medical sensor assembly 102. The sliding engagement locking slots terminate at respective rear wall surfaces 718 and 719.

Disposed on top surface 710 between slot defining portions 712 and 714 are a pair of mutually parallel spaced curved upstanding portions 720 and 722 having curved top edges 724 and 726, respectively, and which define, together with a top edge 728 of forward surface 702, a generally rectangular cut out 730, which extends between upstanding portions 720 and 722 and terminates in a raised platform 732, extending rearwardly of cut out 730 to a location coterminating with the rearward extent of curved upstanding portions 720 and 722.

Turning particularly to FIGS. 7B and 7D, it is seen that the underside/inside of upper housing element 112 includes a pair of mutually parallel upstanding portions 740 and 742 which terminate in mutually axially aligned pivot support edge surfaces 744 and 746, which cooperate with corresponding pivot support cylindrical surfaces 454 and 456 of lower housing element 106 to define pivot supports for generally cylindrical pivot portions 504 and 506 of moveable cover element 108.

Forward of mutually parallel upstanding portions 740 and 742 on opposite sides of rectangular cut out 730 there are formed mutually axially aligned pivot support recesses 754 and 756, which cooperate with corresponding mutually axially aligned pivot support edge surfaces 444 and 446 of lower housing element 106 to provide pivot supports for mutually axially aligned pivot rod portions 604 and 606 of rotatable electrical contact element 114.

Rearward of mutually parallel upstanding portions 740 and 742 there is provided an upstanding portion 760 having a slot 762 for engagement therewith by spring 110 (FIG. 2).

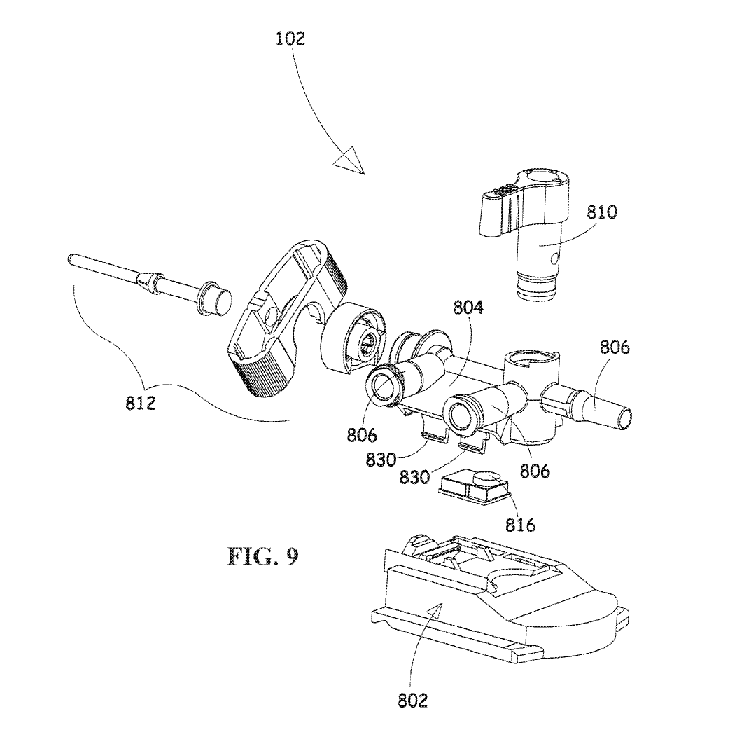

Reference is now made to FIGS. 8A, 8B, 8C and 8D, which are, respectively, simplified pictorial, planar top view, planar bottom view and sectional side view simplified illustrations of a medical sensor assembly, such as medical sensor assembly 102, forming part of the mounting assembly shown in FIGS. 1A-2, the side sectional view being taken along lines D-D in FIG. 8B, and to FIG. 9, which is a simplified exploded view illustration of the medical sensor assembly of FIGS. 8A-8D.

As seen in FIGS. 8A-9, medical sensor assembly 102 comprises a base element 802 and a flowpath defining sensor mounting housing 804, typically having a plurality of ports 806 coupled to a flow channel 808 defined thereby. Housing 804 preferably defines a stopcock body which cooperates with a stopcock handle 810. A flushing subassembly 812 also preferably communicates with the flow channel 808, and is preferably identical or similar to that shown and described with reference to FIG. 6 of U.S. Pat. No. 6,511,434, the disclosure of which is hereby incorporated by reference.

Housing 804 includes a pressure sensor location 814, in which is mounted a pressure sensor 816, such as a Measurement 1620 sensor, commercially available from Measurement Specialties, Hampton, Va., USA. Pressure sensor 816 is preferably mounted in operative engagement with the flow channel 808, as seen in FIG. 8D. As distinguished from the device described in U.S. Pat. No. 6,511,434, electrical contact pads 820 of pressure sensor 816 are available for removable direct galvanic contact engagement with electrical contact pins 620 of rotatable electrical contact element 114. Furthermore, as seen in FIG. 8C and as distinguished from the teachings of U.S. Pat. No. 6,511,434, the electric contact pads 820 of pressure sensor 816 are preferably exposed.

Housing 804 preferably defines a plurality of mounting protrusions 830 for mounting thereof onto base element 802.

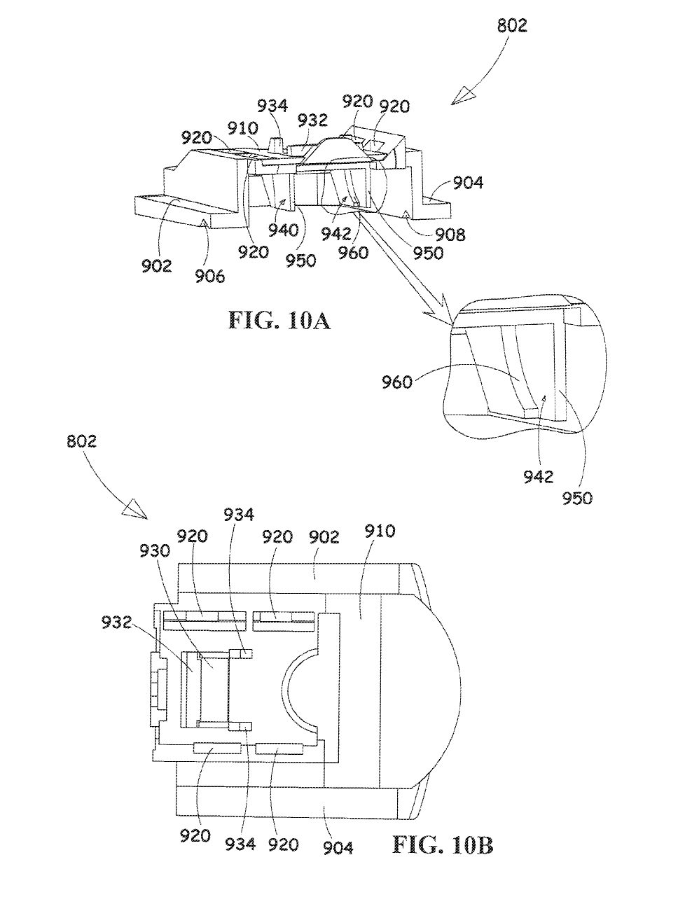

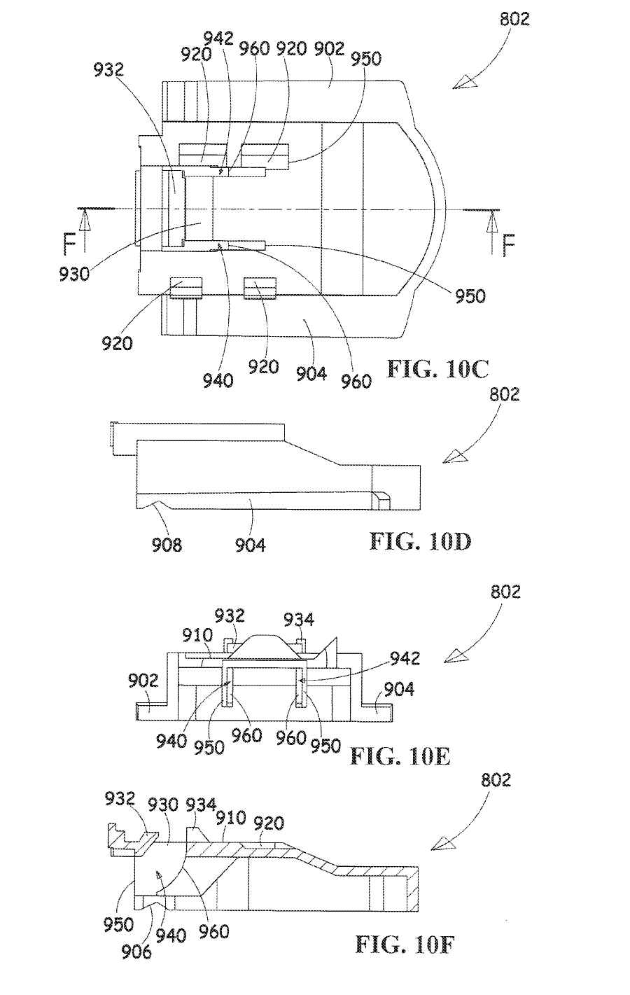

Reference is now made to FIGS. 10A, 10B, 10C, 10D, 10E and 10F, which are, respectively, simplified pictorial, planar bottom view, planar top view, planar side view, planar end view and internal side sectional view illustrations of a base element, such as base element 802, forming part of the medical sensor assembly shown in FIGS. 1A-2, 8A-8D and 9, the internal side sectional view being taken along lines F-F in FIG. 10C.

As seen in FIGS. 10A-10F, base element 802 preferably comprises a pair of mutually parallel, mutually spaced downward facing elongate sliding surfaces 902 and 904 which are configured for slidable engagement with generally planar outer facing top surface 710 of upper housing element 112 along a longitudinal sliding pathway defined by respective generally longitudinal upwardly extending and inwardly transversely extending slot defining portions 712 and 714 of upper housing element 112.

Base element 802 defines a generally planar top facing surface 910 located above and between elongate sliding surfaces 902 and 904. Elongate sliding surfaces 902 and 904 are formed with respective recesses 906 and 908 configured for snap engagement with corresponding protrusions 717 and 716 of upper housing element 112. Top facing surface 910 is preferably formed with a plurality of mounting apertures 920 for accommodating mounting apertures 830 of housing 804 in a locked snap fit arrangement. Top facing surface 910 is also formed with an aperture 930 for providing engagement access to electrical contact pads 820 of pressure sensor 816 by electrical contact pins 620 of rotatable electrical contact element 114 for direct removable galvanic contact engagement therewith.

A tapered built up edge 932 is formed on top facing surface 910 alongside aperture 930 for providing a guiding surface for rotatable electrical contact element 114 during sliding engagement between the medical sensor assembly 102 and the mounting assembly 100. Additionally, two protrusions 934 are located alongside aperture 930 opposite edge 932, for supporting the rotatable electrical contact element 114 in a desired raised orientation for direct galvanic contact engagement with electrical contact pads 820 of pressure sensor 816.

Turning now particularly to the underside of base element 802, as seen particularly in FIGS. 10C & 10F, it is seen that there are preferably provided a pair of mutually parallel, mutually spaced upstanding portions 940 and 942. It is a particular feature of the present invention that mutually parallel, mutually spaced upstanding portions 940 and 942 define cam surfaces which, upon sliding engagement of the medical sensor assembly 102 with the mounting assembly 100, provide coordinated rotation of moveable cover element 108 and of rotatable electrical contact element 114.

In accordance with a preferred embodiment of the present invention, each of mutually spaced upstanding portions 940 and 942 defines a cam surface defining edge 950, which is operative to engage a portion of edge portion 540 of moveable cover element 108 and cause moveable cover element 108 to rotate about an axis defined by pivot portions 504 and 506, thereby to provide access to electric contact pins 620 of rotatable electrical contact element 114.

Further in accordance with a preferred embodiment of the present invention, each of mutually spaced upstanding portions 940 and 942 is configured to define along a mutually facing side wall portion thereof a curved cam surface 960, which is operative to engage a finger 618 of rotatable electrical contact element 114 and cause rotatable electrical contact element 114 to rotate about an axis defined by pivot portions 604 and 606, thereby to position electric contact pins 620 of rotatable electrical contact element 114 in galvanic electrical engagement with pads 820 of sensor 816. It is also a particular feature of the present invention that the rotations of the moveable cover element 108 and of the rotatable electrical contact element 114 are precisely coordinated in their sequence by virtue of the definition of both cam surfaces 950 and 960 on the same portion.

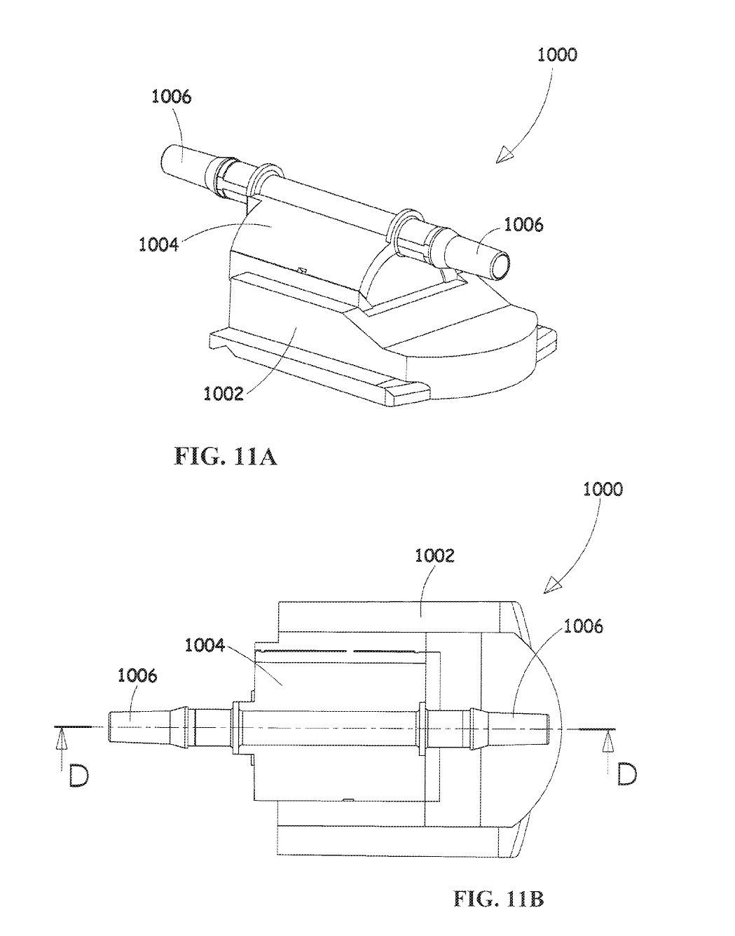

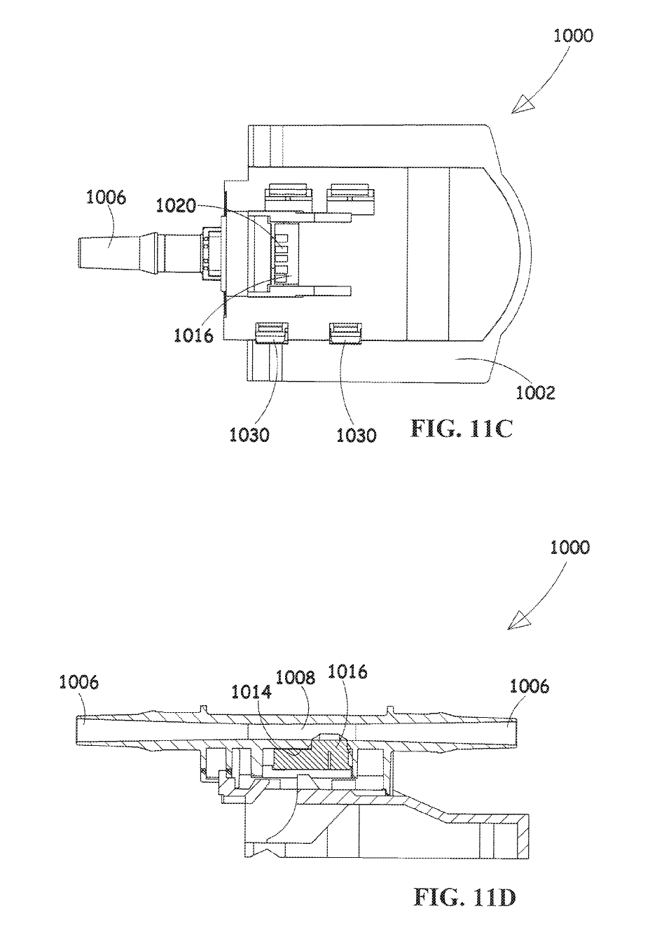

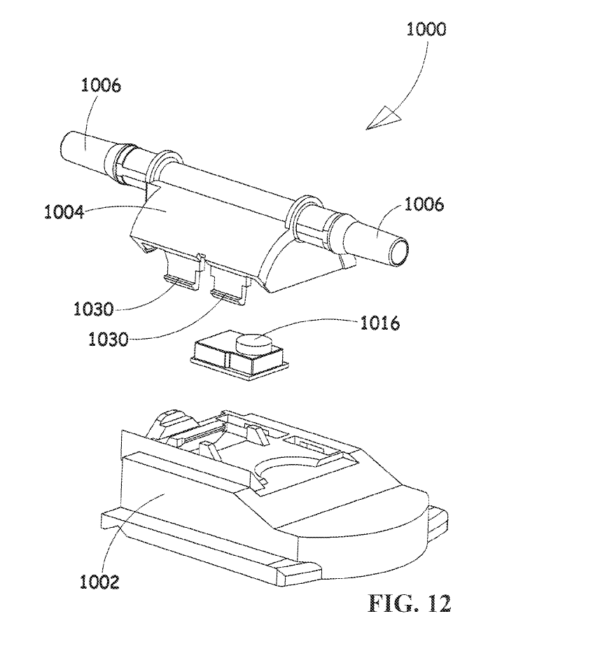

Reference is now made to FIGS. 11A, 11B, 11C and 11D, which are, respectively, pictorial, planar top view, planar bottom view and sectional side view simplified illustrations of a medical sensor assembly, forming part of the mounting assembly shown in FIGS. 1A-2, the side sectional view being taken along lines D-D in FIG. 11B, and to FIG. 12, which is a simplified exploded view illustration of the medical sensor assembly of FIGS. 11A-11D.

As seen in FIGS. 11A-12, a medical sensor assembly 1000 comprises a base element 1002, preferably identical to base element 802 described hereinabove, and a flowpath defining sensor mounting housing 1004, typically having two ports 1006 coupled to a flow channel 1008 defined thereby.

Housing 1004 includes a pressure sensor location 1014, in which is mounted a pressure sensor 1016, such as a Measurement 1620 sensor, commercially available from Measurement Specialties, Hampton, Va., USA. Pressure sensor 1016 is preferably mounted in operative engagement with flow channel 1008, as seen in FIG. 11D. As distinguished from the device described in U.S. Pat. No. 6,511,434, electrical contact pads 1020 of pressure sensor 1016 are available for removable direct galvanic contact engagement with electrical contact pins 620 of rotatable electrical contact element 114. Furthermore as seen in FIG. 11C and as distinguished from the teachings of U.S. Pat. No. 6,511,434, the electric contact pads 1020 of pressure sensor 1016 are preferably exposed.

Housing 1004 preferably defines a plurality of mounting protrusions 1030 for mounting thereof onto base element 1002.

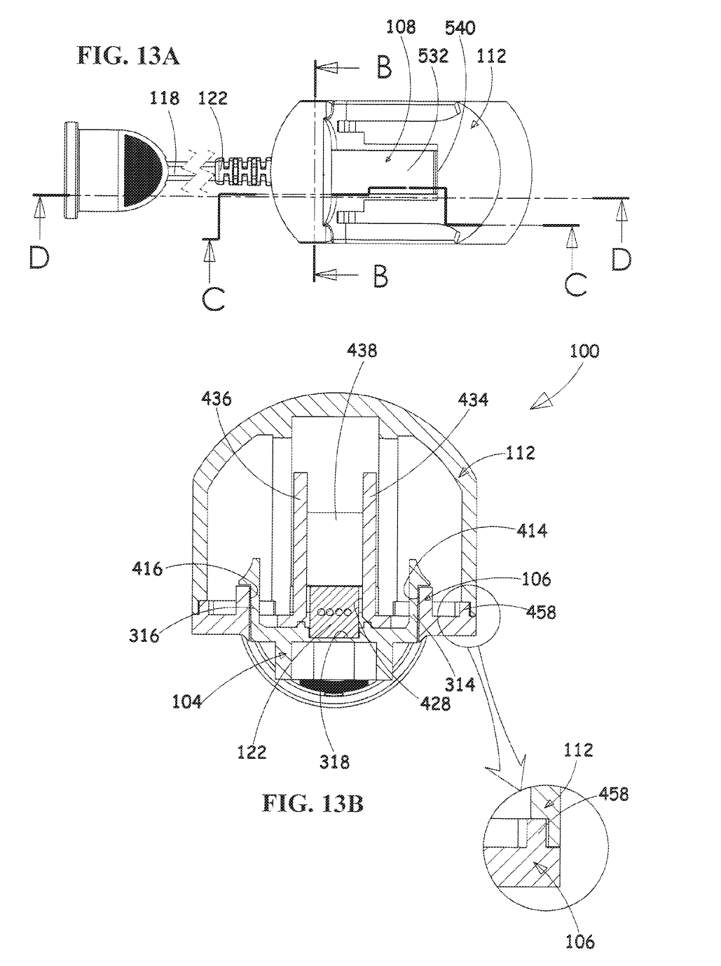

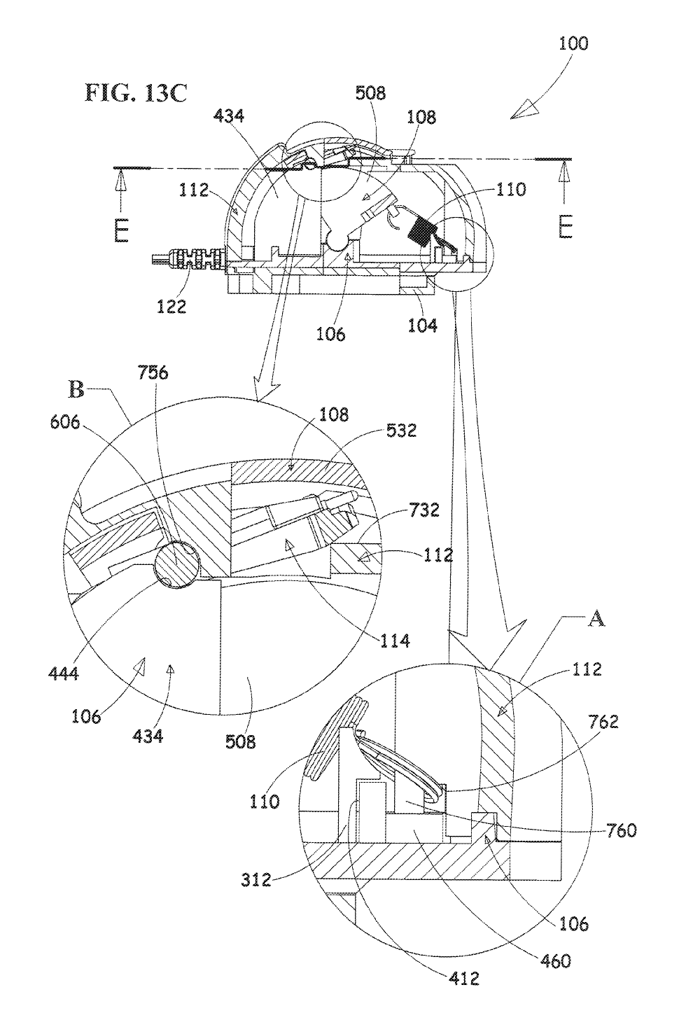

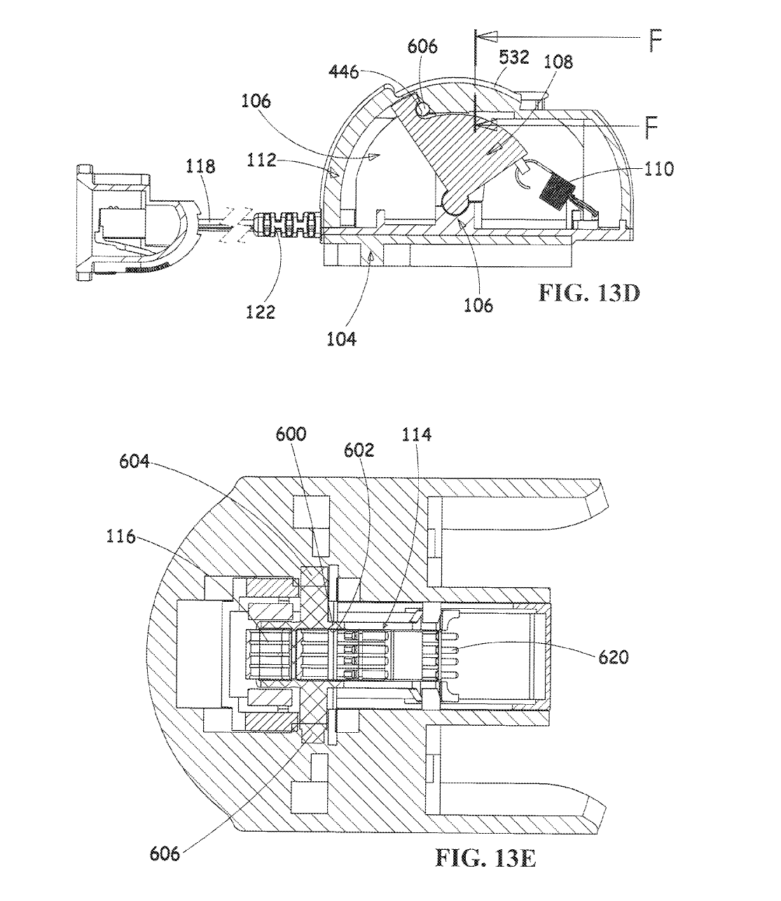

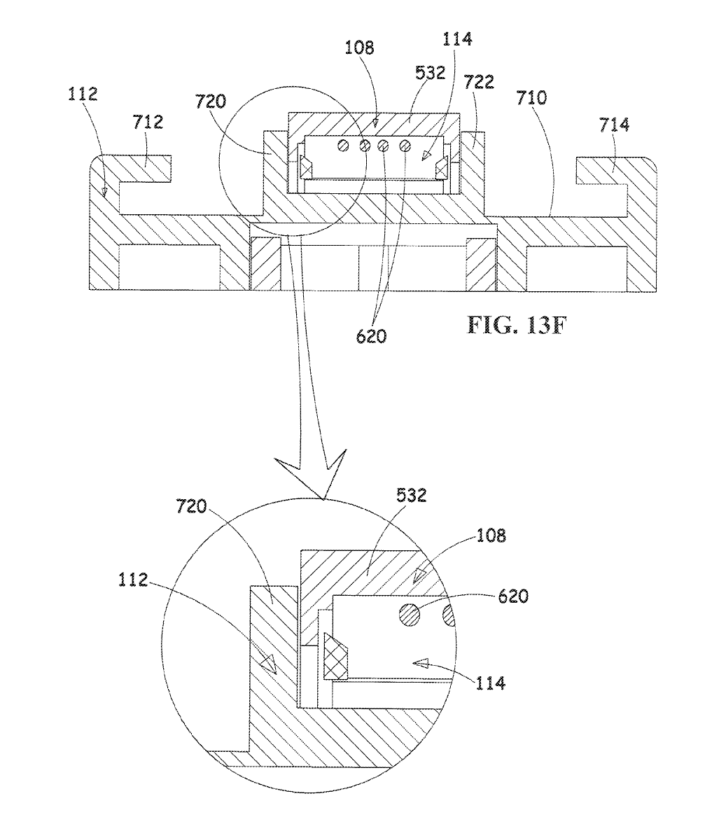

Reference is now made to FIGS. 13A, 13B, 13C, 13D, 13E and 13F, which are respectively, a planar top view and five sectional simplified illustrations of the mounting assembly 100 of FIGS. 1A-2, the sectional illustrations being taken along respective lines B-B, C-C and D-D in FIG. 13A, lines E-E in FIG. 13C and lines F-F in FIG. 13D. FIGS. 13A-13F show the mounting assembly 100 when it is not engaged by a medical sensor assembly.

FIG. 13B shows the locking engagement between base element 104 and lower housing element 106. Specifically, the snap fit engagement between engagement protrusions 314 and 316 and respective throughgoing apertures 414 and 416 is shown, as is the tight fit engagement between lower housing element 106 and upper housing element 112 at circumferential protrusion 458 of the lower housing element. Preferably the lower housing element 106 and the upper housing element 112 are ultrasonically welded to each other at discrete locations along circumferential protrusion 458. The seating of electrical contact end portion 122 in monitor connection cord end retaining recess 318 of base element 104 and in throughgoing cutout 428 of lower housing element 106 is also seen.

FIG. 13C shows, particularly at enlargement A, the locking engagement of an end of spring 110 in slot 762 of upper housing element 112 which is also defined by spring attachment protrusion 460 of lower housing element 106. The snap fit engagement between engagement protrusion 312 throughgoing apertures 412 is also seen in enlargement A.

Enlargement B shows the arrangement of the rotatable electrical contact element 114 underlying and covered by the curved cover portion 532 of moveable cover element 108. It is seen that a forward end of rotatable electrical contact element 114 lies on an edge of raised platform 732.

As seen in Enlargement B, pivot rod portions 604 and 606 are rotatably retained between respective support edge surfaces 444 and 446 of lower housing element 106 and respective pivot support recesses 754 and 756 of upper housing element 112.

FIG. 13D shows the operative engagement of spring 110 with moveable cover element 108.

FIG. 13E illustrates details of the rotatable electrical contact element 114 including base element 600, generally rectangular portion 602 having extending outwardly therefrom and transversely thereto pivot rod portions 604 and 606, which are seen to be arranged for pivotable rotation while being supported by respective mutually axially aligned pivot support edge surfaces 444 and 446. FIG. 13E also shows that electrical contact pins 620 are soldered to conductors of flexible connector 116.

FIG. 13F shows the general sealing engagement between curved cover portion 532 of moveable cover element 108 and curved upstanding portions 720 and 722.

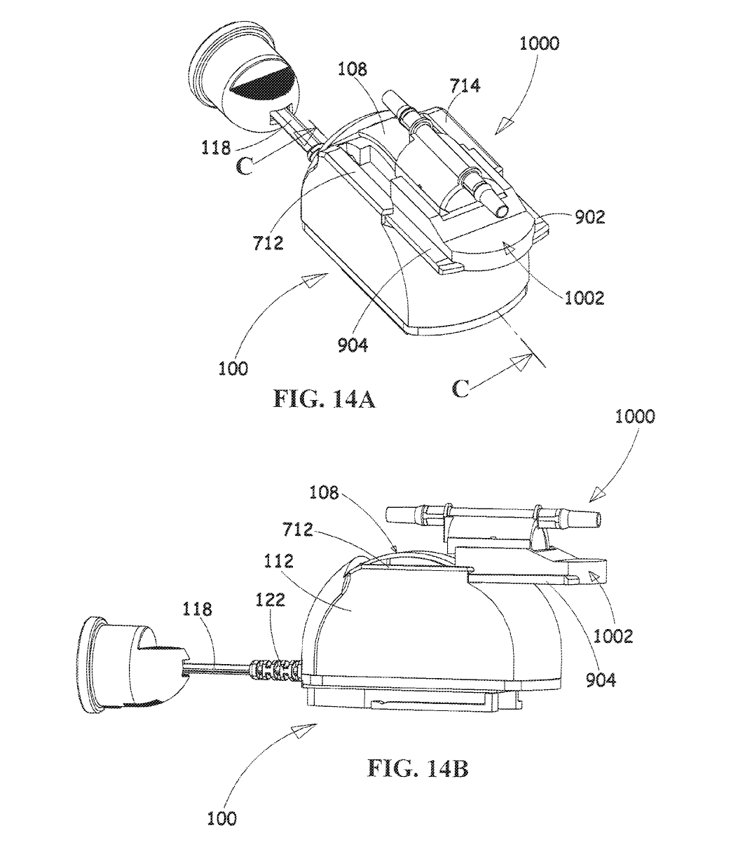

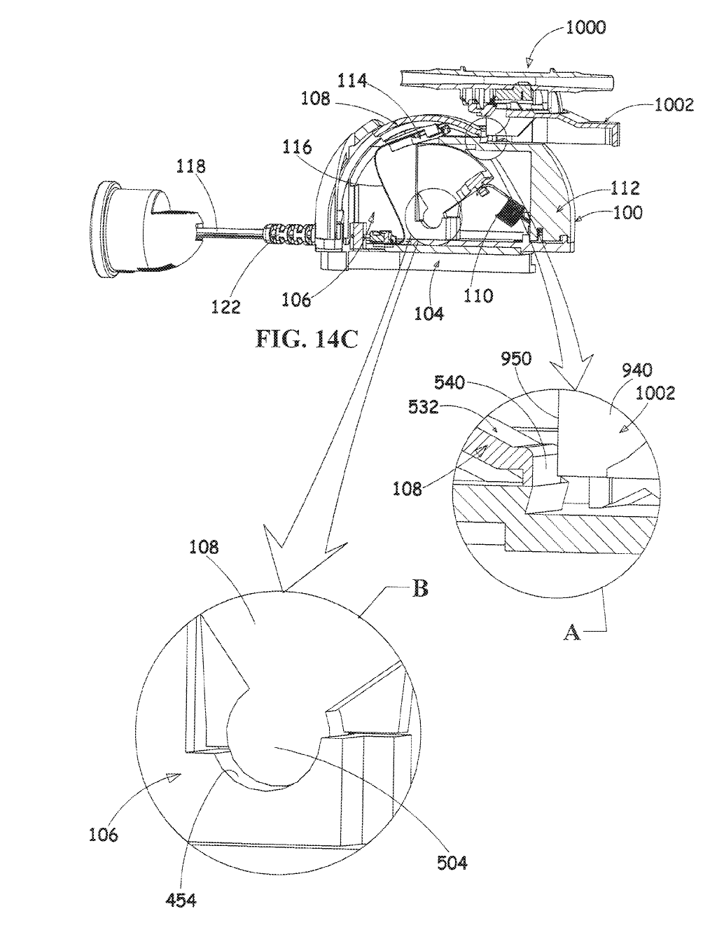

Reference is now made to FIGS. 14A and 14B, which are simplified pictorial top view and side view illustrations of the medical sensor assembly of FIGS. 11A-13 in a first operative orientation, and to FIG. 14C, which is a simplified sectional illustration of the medical sensor assembly of FIGS. 11A-14B in a first operative orientation taken along section lines C-C in FIG. 14A.

As seen in FIGS. 14A-14C, in the first operative orientation, the medical sensor assembly 1000 is at the beginning of a linear sliding engagement movement with respect to mounting assembly 100 wherein forward portions of elongate sliding surfaces 902 and 904 are located in corresponding rearward portions of slots defined by slot defining portions 712 and 714 respectively.

As seen in enlargement A of FIG. 14C, cam surface defining edges 950 of mutually spaced upstanding portions 940 and 942 of base element 1002 of medical sensor assembly 1000 are in touching engagement with edge portion 540 of moveable cover element 108 but have not yet displaced the moveable cover element 108. Moveable cover element 108 is in its fully closed orientation and is retained in this orientation by spring 110, which is slightly tensioned. As seen in enlargement B of FIG. 14C, moveable cover element 108 is pivotably mounted by its cylindrical pivot portions 504 and 506 onto pivot support cylindrical surfaces 454 and 456 of lower housing element 106.

As further seen in FIG. 14C, in the first operative orientation, the rotatable electrical contact element 114 is in a first orientation.

At this stage and throughout the linear sliding engagement of the medical sensor assembly 1000 with the mounting assembly 100, elongate sliding surfaces 902 and 904 lie generally parallel to corresponding slot defining portions 712 and 714.

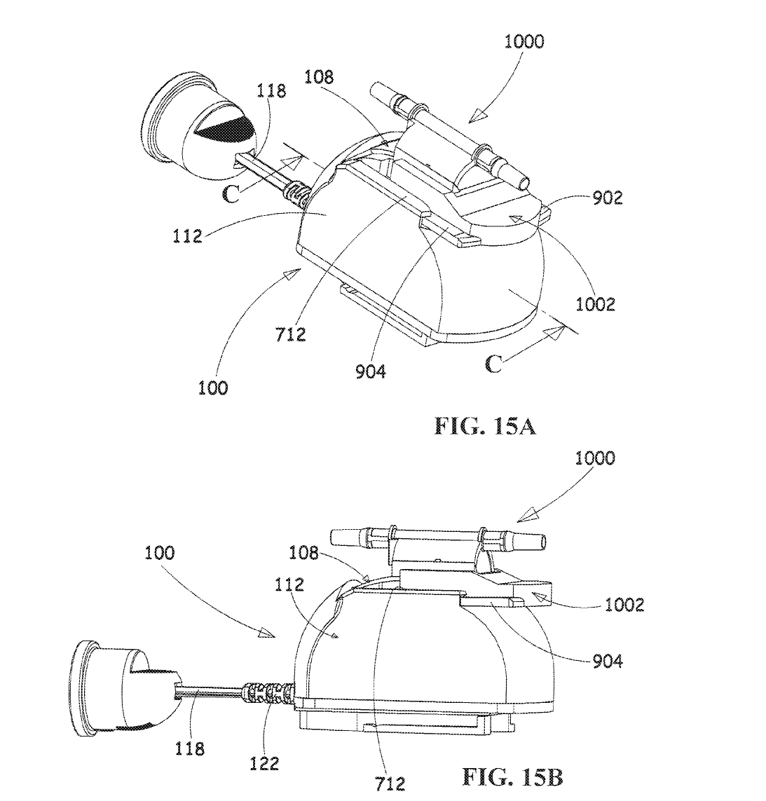

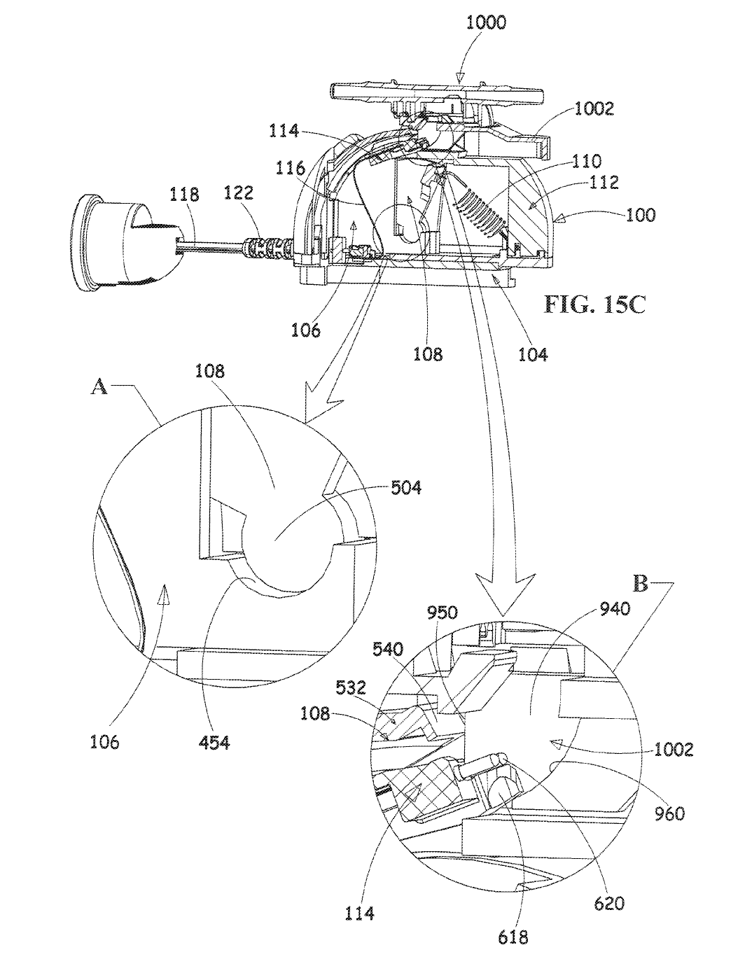

Reference is now made to FIGS. 15A and 15B, which are simplified pictorial top view and side view illustrations of the medical sensor assembly of FIGS. 11A-14D in a second operative orientation, and to FIG. 15C, which is a simplified sectional illustration of the medical sensor assembly of FIGS. 11A-15B in the second operative orientation, taken along section lines C-C in FIG. 15A.

As seen in FIGS. 15A-15C, in the second operative orientation, the medical sensor assembly 1000 is partially along its linear sliding engagement movement with respect to mounting assembly 100 wherein forward and intermediate portions of elongate sliding surfaces 902 and 904 are located in corresponding rearward and intermediate portions of slots defined by slot defining portions 712 and 714 respectively.

Cam surface defining edges 950 of mutually spaced upstanding portions 940 and 942 of base element 1002 of medical sensor assembly 1000 are in touching, cam driving engagement with edge portion 540 of moveable cover element 108 and have partially displaced the moveable cover element 108 along a rotatable path defined by rotational engagement of cylindrical pivot portions 504 and 506 with pivot support cylindrical surfaces 454 and 456 of lower housing element 106 against the urging of spring 110.

Moveable cover element 108 is in a partially open orientation. As seen in enlargement A of FIG. 15C, moveable cover element 108 has pivoted counterclockwise relative to its position as seen in enlargement A of FIG. 14C.

In the second operative orientation, the rotatable electrical contact element 114 remains in a generally first orientation, as seen best in enlargement B of FIG. 15C, since sensor engagement fingers 618 are not yet in operative engagement with curved cam surfaces 960.

At this stage and throughout the linear sliding engagement of the medical sensor assembly 102 with the mounting assembly 100, elongate sliding surfaces 902 and 904 lie generally parallel to corresponding slot defining portions 712 and 714.

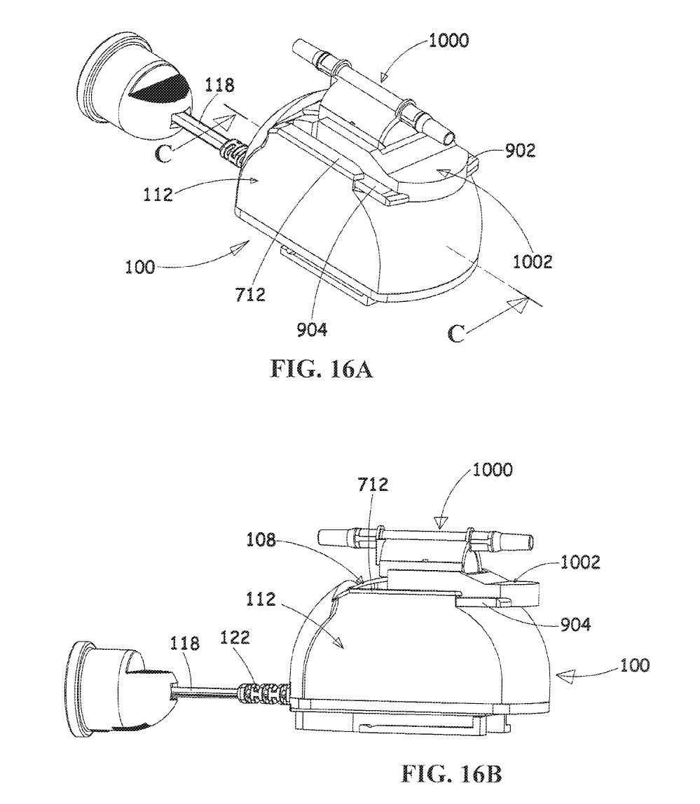

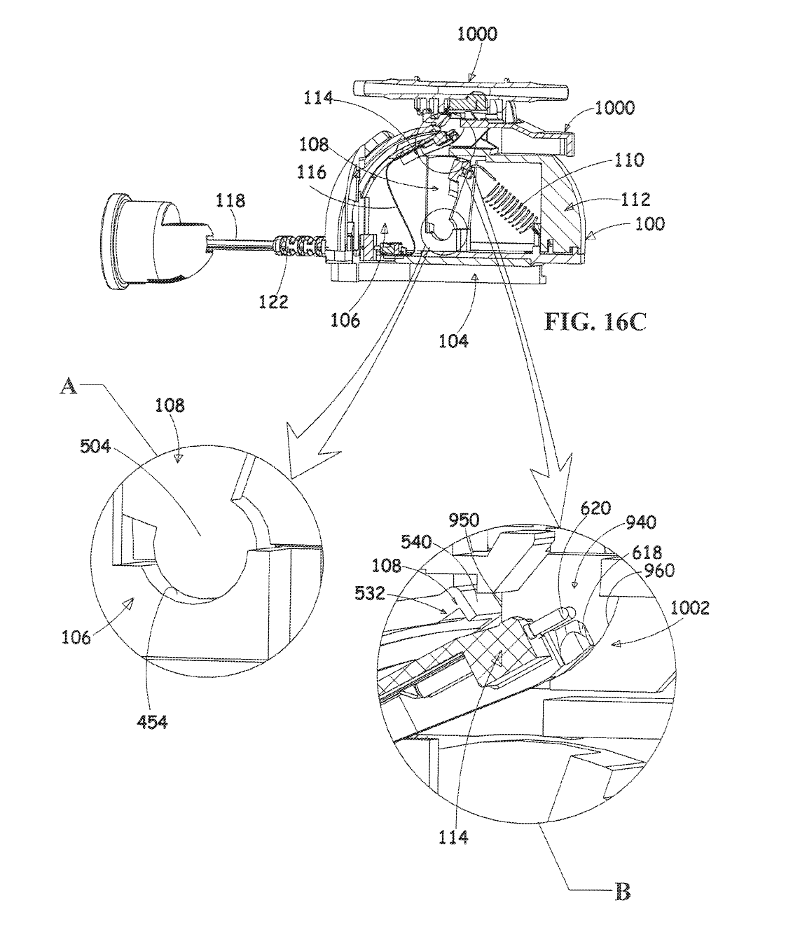

Reference is now made to FIGS. 16A and 16B, which are simplified pictorial top view and side view illustrations of the medical sensor assembly of FIGS. 11A-15D in a third operative orientation, and to FIG. 16C, which is a simplified sectional illustration of the medical sensor assembly of FIGS. 11A-16B in the third operative orientation, taken along section lines C-C in FIG. 16A.

As seen in FIGS. 16A-16C, in the third operative orientation, the medical sensor assembly 1000 is further along its linear sliding engagement movement with respect to mounting assembly 100 wherein nearly all portions of elongate sliding surfaces 902 and 904 are located in nearly all corresponding portions of slots defined by slot defining portions 712 and 714, respectively.

Cam surface defining edges 950 of mutually spaced upstanding portions 940 and 942 of base element 1002 of medical sensor assembly 1000 are in touching, cam driving engagement with edge portion 540 of moveable cover element 108 and have nearly fully displaced the moveable cover element 108 along a rotatable path defined by rotational engagement of cylindrical pivot portions 504 and 506 with pivot support cylindrical surfaces 454 and 456 of lower housing element 106 against the urging of spring 110.

Moveable cover element 108 is in a nearly fully open orientation. As seen in enlargement A of FIG. 16C, moveable cover element 108 has pivoted counterclockwise relative to its position as seen in enlargement A of FIG. 16C.

In the third operative orientation, the rotatable electrical contact element 114 is now in an inclined orientation, as seen best in enlargement B of FIG. 16C, as the result of operative engagement between sensor engagement fingers 618 and curved cam surfaces 960. It is noted, however that the electric contact pins 620 are not yet in direct galvanic contact with pads 1020 of sensor 1016 of the medical sensor assembly 1000. It is noted that flexible connector 116 is somewhat repositioned in order to accommodate the repositioning of rotatable electrical contact element 114.

At this stage and throughout the linear sliding engagement of the medical sensor assembly 1000 with the mounting assembly 100, elongate sliding surfaces 902 and 904 lie generally parallel to corresponding slot defining portions 712 and 714.

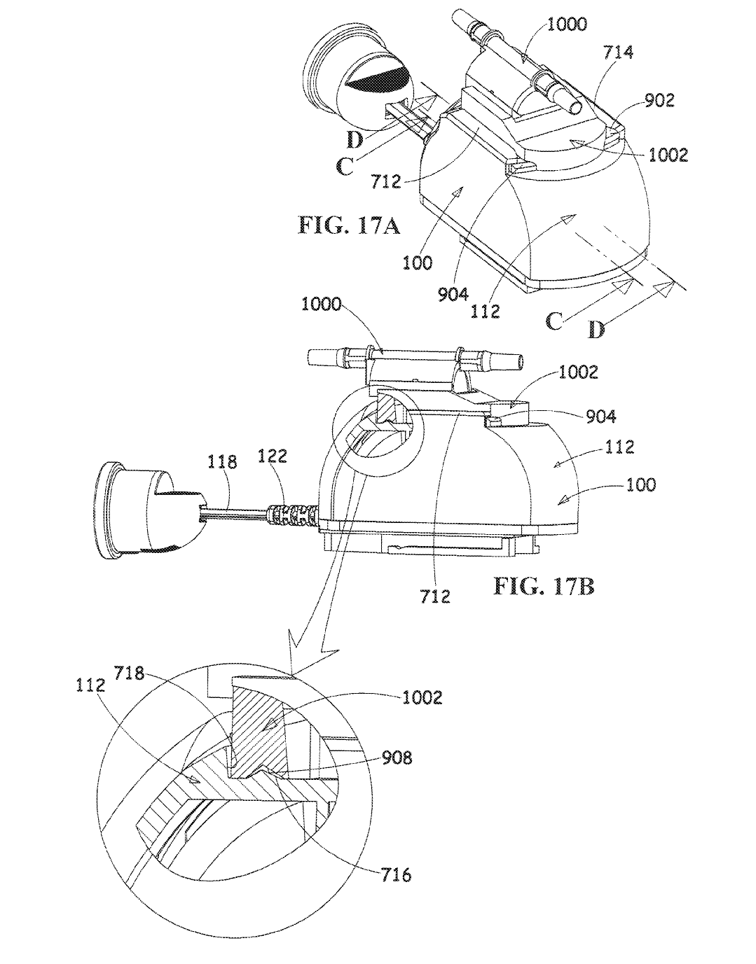

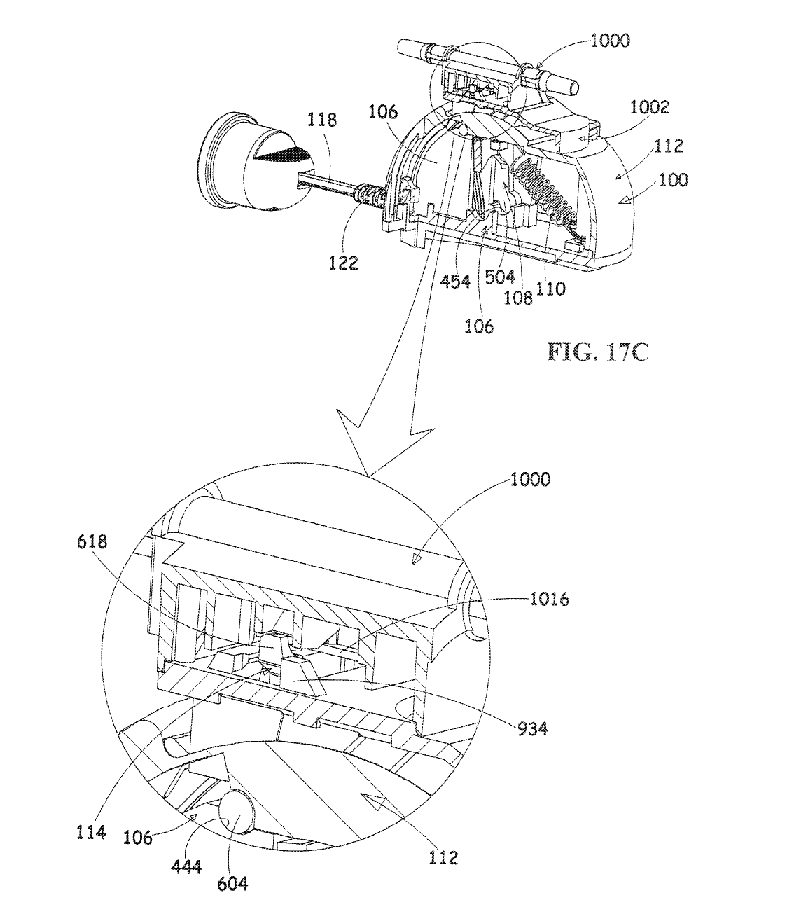

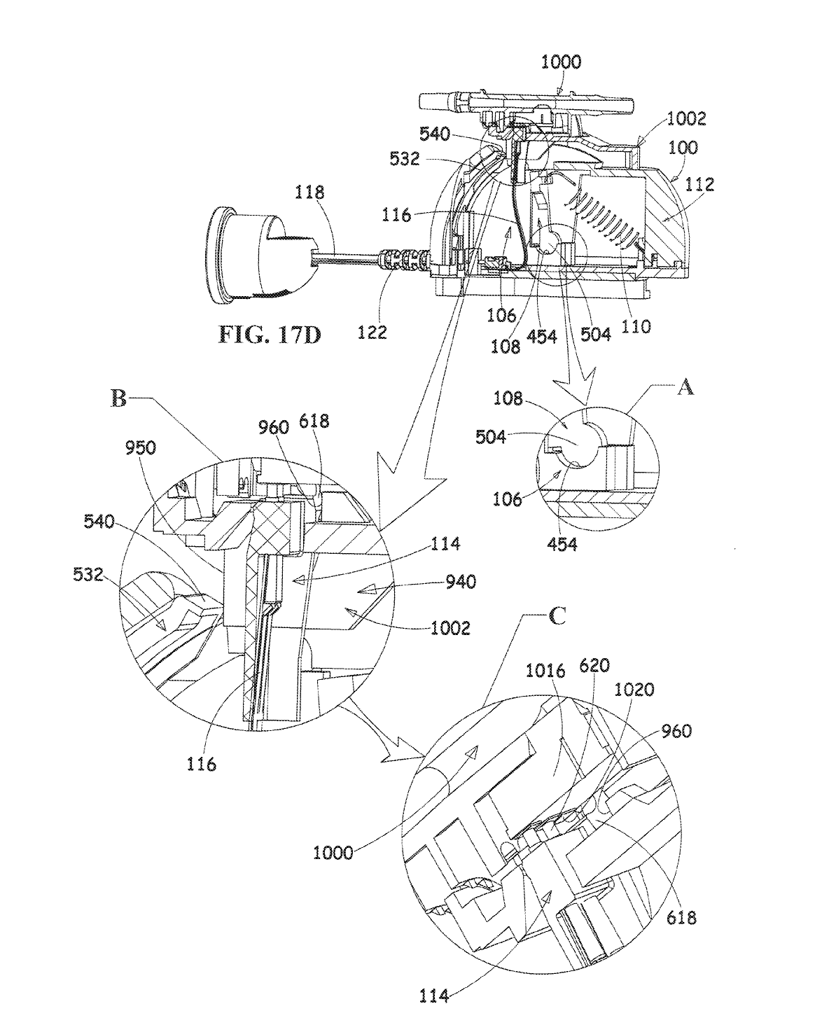

Reference is now made to FIGS. 17A and 17B, which are simplified pictorial top view and side view illustrations of the medical sensor assembly of FIGS. 11A-16D in a fourth operative orientation, and to FIGS. 17C and 17D, which are simplified sectional illustrations of the medical sensor assembly of FIGS. 11A-17B in the third operative orientation, taken along respective section lines C-C and D-D in FIG. 17A.

As seen in FIGS. 17A-17D, in the fourth operative orientation, the medical sensor assembly 1000 is at the end of its linear sliding engagement movement with respect to mounting assembly 100 wherein elongate sliding surfaces 902 and 904 are fully engaged with corresponding portions of slots defined by slot defining portions 712 and 714 respectively. This fourth operative orientation is preferably defined by engagement of rear wall surfaces 718 and 719 of upper housing element 112 by the forward edge of base element 1002, which defines a linear travel stop. The engagement of the medical sensor assembly 1000 with the mounting assembly 100 in the fourth operative orientation is a snap fit engagement produced by engagement of raised protrusions 716 and 717 with corresponding recesses 908 and 906 in base element 1002.

Cam surface defining edges 950 of mutually spaced upstanding portions 940 and 942 of base element 1002 of medical sensor assembly 1000 are in touching, cam driving engagement with edge portion 540 of moveable cover element 108 and have fully displaced the moveable cover element 108 along a rotatable path defined by rotational engagement of cylindrical pivot portions 504 and 506 with pivot support cylindrical surfaces 454 and 456 of lower housing element 106 against the urging of spring 110.

Moveable cover element 108 is in a fully open orientation. As seen in enlargement A of FIG. 17D, moveable cover element 108 has further pivoted counterclockwise relative to its position as seen in enlargement A of FIG. 16C.

In the fourth operative orientation, the rotatable electrical contact element 114 is now in a second orientation, as seen best in enlargements B and C of FIG. 17D, as the result of operative engagement between sensor engagement fingers 618 and curved cam surfaces 960. It is noted, that the electric contact pins 620 are now in direct galvanic contact with pads 1020 of sensor 1016 of the medical sensor assembly 1000. It is noted that flexible connector 116 is further repositioned in order to accommodate the repositioning of rotatable electrical contact element 114.

It is appreciated that electric contact pins 620 and pads 1020 of sensor 1016 are in direct galvanic engagement only when medical sensor assembly 1000 is mounted on mounting assembly 100 and is in the fourth operating orientation shown in FIGS. 17A-17D.

At this stage and throughout the linear sliding engagement of the medical sensor assembly 102 with the mounting assembly 100, elongate sliding surfaces 902 and 904 lie generally parallel to corresponding slot defining portions 712 and 714.

Upon disengagement of the medical sensor assembly 102 from the mounting assembly 100, moveable cover element 108 returns, under the urging of spring 110, to the closed position described hereinabove with reference to FIGS. 13A-13F.

It is appreciated that in the closed position, moveable cover element 108 covers and protects rotatable electrical contact element 114 during swabbing or other cleaning of the outside of mounting assembly 100 following use thereof, such that application of liquids to the mounting assembly does not result in shorting of electrical contacts of the rotatable electrical contact element 114 by such liquid.

It will be appreciated by persons skilled in the art that the present invention is not limited by what has been particularly shown and described hereinabove. Rather the scope of the present invention includes combinations and subcombinations of features described above as well as modifications and improvements thereof that are not in the prior art.

1. A pressure transducer assembly comprising:
a mounting assembly including an electrical connector configured for selectable galvanic engagement with a pressure transducer; and
a medical sensor assembly including a pressure transducer comprising:
a flow pathway; and
a pressure sensor in pressure sensing communication with said flow pathway, said pressure sensor including a plurality of electrical contacts configured for direct galvanic engagement with said electrical connector only when said medical sensor assembly is mounted in operative engagement with said mounting assembly.

2. A pressure transducer assembly according to claim 1 and wherein said electrical connector is configured to be moved by engagement with said medical sensor assembly from a first non-electrically connected operative orientation to a second electrically connected operative orientation.

3. A pressure transducer assembly according to claim 2 and wherein said medical sensor assembly also comprises an electrical connector engagement element operative to move said electrical connector from said first non-electrically connected operative orientation to said second electrically connected operative orientation upon engagement of said medical sensor assembly with said mounting assembly.

4. A pressure transducer assembly according to claim 1 and also comprising a cover element for preventing fluid contact with said electrical connector when said mounting assembly is not in engagement with said medical sensor assembly.

5. A pressure transducer assembly according to claim 4 and wherein:
said mounting assembly also comprises an upper housing portion; and
said cover element is in a protective position with respect to said upper housing portion thereby preventing said fluid contact with said electrical connector when said mounting assembly is not in engagement with said medical sensor assembly.

6. A pressure transducer assembly according to claim 1 and wherein said medical sensor assembly slidingly engages said mounting assembly to move said electrical connector from said first non-electrically connected operative orientation to said second electrically connected operative orientation.

7. A pressure transducer assembly according to claim 1 and wherein said plurality of electrical contacts of said pressure sensor are exposed when said medical sensor assembly is not engaged with said mounting assembly.

8. A pressure transducer assembly according to claim 1 and wherein said medical sensor assembly includes an aperture for providing access to said plurality of electrical contacts of said pressure sensor by said electrical connector for said selectable direct galvanic engagement.

9. A medical sensor assembly useful with a mounting assembly having an electrical connector arranged for selectable galvanic engagement with said medical sensor assembly, said medical sensor assembly comprising:
a pressure transducer comprising:
a flow pathway; and
a pressure sensor in pressure sensing communication with said flow pathway, said pressure sensor including a plurality of exposed electrical contact pads configured for direct galvanic engagement with said electrical connector when said medical sensor assembly is mounted in operative engagement with said mounting assembly.

10. A medical sensor assembly according to claim 9 and wherein said exposed electrical contact pads are configured for direct galvanic engagement with said electrical connector only when said medical sensor assembly is mounted in operative engagement with said mounting assembly.

11. A medical sensor assembly according to claim 9 and wherein said electrical connector is configured to be moved by engagement with said medical sensor assembly from a first non-electrically connected operative orientation to a second electrically connected operative orientation.

12. (canceled)

13. A medical sensor assembly according to claim 11 and wherein said medical sensor assembly slidingly engages said mounting assembly to move said electrical connector from said first non-electrically connected operative orientation to said second electrically connected operative orientation.

14. A medical sensor assembly according to claim 9 and wherein said medical sensor assembly includes an aperture for providing access to said plurality of exposed electrical contact pads of said pressure sensor by said electrical connector for said selectable direct galvanic engagement.

15. A mounting assembly for use with a medical sensor assembly, said medical sensor assembly including a pressure transducer, said mounting assembly comprising:
an electrical connector configured for selectable direct galvanic engagement with said pressure transducer; and
a cover element for preventing fluid contact with said electrical connector when said mounting assembly is not in engagement with said medical sensor assembly.

16. A mounting assembly according to claim 15 and also comprising an upper housing portion which cooperates with said cover element, said cover element being in electrical connector protective engagement in cooperation with said upper housing portion when said mounting assembly is not in engagement with said medical sensor assembly.

17. A mounting assembly according to claim 15 and wherein said electrical connector is configured to be moved by engagement with said medical sensor assembly from a first non-electrically connected operative orientation to a second electrically connected operative orientation.

18. A mounting assembly according to claim 15 and wherein said mounting assembly is configured for sliding engagement with said medical sensor assembly.

19. A method for removably interconnecting a pressure transducer assembly and a monitor, said method comprising:
providing a pressure transducer assembly including a medical sensor assembly and a mounting assembly, said medical sensor assembly including a pressure transducer having a plurality of electrical contacts, said mounting assembly including a first electrical connector configured for selectable direct galvanic engagement with said plurality of electrical contacts and a second electrical connector arranged to be connected to said monitor and to said first electrical connector;
positioning said mounting assembly onto a fixed support;
electrically connecting said second electrical connector with said monitor; and
mounting said medical sensor assembly onto said mounting assembly, such that said plurality of electrical contacts of said pressure transducer automatically establish direct galvanic engagement with said first electrical connector when said medical sensor assembly is mounted in operative engagement with said mounting assembly.

20. A method according to claim 19 and also comprising moving said first electrical connector from a first non-electrically connected operative orientation to a second electrically connected operative orientation.

21. (canceled)

22. A method according to claim 19 and wherein said mounting said medical sensor assembly onto said mounting assembly comprises linearly displacing said medical sensor assembly into engagement with said mounting assembly.
